(19) United States
(12) Patent Application Publication
(10) Pub. No.: US 2005/0172852 A1
(43) Pub. Date: Aug. 11, 2005

(54) Variable appearance tissue markings

(75) Inventors: Richard Rox Anderson, Lexington, MA (US); Susanna K. Mlynarczyk-Evans, San Francisco, CA (US); Craig A. Drill, New York, NY (US)

Correspondence Address: FISH & RICHARDSON PC, 225 FRANKLIN ST, BOSTON, MA, 02110, US

(73) Assignees: The General Hospital Corporation, Boston, MA (US); Freedom-2, LLC, New York, NY (US)

(21) Appl. No.: 10/987,702

(22) Filed: Nov. 12, 2004

Related U.S. Application Data
(60) Provisional application No. 60/519,459, filed on Nov. 12, 2003.

(51) Int. Cl. C09D 1/00; C09D 4/00; C09D 5/00; A61F 13/00

(52) U.S. Cl. 106/31.03; 424/422

(57) ABSTRACT

The present invention provides particles that create permanent tissue markings, including, but not limited to, tattoos, that have variable appearance properties. Also, the present invention provides methods for producing, implanting, altering and removing these variable appearance tissue markings.

### CLAIM OF PRIORITY

This application claims priority under 35 USC § 119(e) to U.S. Patent Application Ser. No. 60/519,459, filed on Nov. 12, 2003, the entire contents of which are hereby incorporated by reference.

### FIELD OF THE INVENTION

The invention relates to permanent, variable appearance tissue markings, including, but not limited to, tattoos. Also, the present invention provides methods for producing, implanting, altering and removing these tissue markings.

### BACKGROUND OF THE INVENTION

Tattoos have been used in almost every culture throughout history. They have been found on a five thousand year old human mummy, and decorated figurines suggest their use at least fifteen thousand years ago. Tattoos have been used for many purposes including identity, beauty, artistic and spiritual expression, medicine, and magic.

In the United States, statistics are not kept on tattooing, but the practice has apparently been growing in popularity for the past few decades. The majority of tattoos are apparently obtained by people under forty years of age, including a significant proportion of teenagers. An estimated 2 million people are tattooed every year.

In the United States today, tattoo uses include not only the familiar artistic tattoo, but also permanent makeup (for example, permanent eyebrows, eyeliner, lip liner, and lip color); corrective or reconstructive pigmentation (for example, repigmentation of scar tissue or areola reconstruction on mastectomy patients); medical markings (for example, marking gastrointestinal surgery sites for future monitoring or marking locations for radiation treatment); and identification markings on animals (for example, pedigree “tags” on purebred pets).

The tattooing procedure consists of piercing the skin or other tissue with needles or similar instruments to introduce an ink that includes small particles of pigment suspended in a liquid carrier. During the healing process, some particles of pigment are sloughed from the skin surface and others are transported to the lymphatic system. What one sees as the tattoo are the remaining particles of pigment located in the dermis where they are engulfed by phagocytic skin cells (such as fibroblasts and macrophages) or are retained in the extracellular matrix.

Tattoos typically consist of micrometer or submicrometer-scale, inert and insoluble particles, which are retained in the dermis layer of skin. To create a permanent tattoo, for example, one must implant pigments that are not dissolved or digested by living tissue. Alternatively, dispersible pigments may be encapsulated in appropriately sized particles, such as those disclosed in U.S. Pat. No. 6,013,122. These particles can be designed so that they may be rendered invisible by applying a specific form of energy.

Primitive pigments probably consisted of graphite and other carbon substances. Modern pigments include inorganic metal salts and brightly colored organometallic complexes.

Typically, tattoos are visible under normal lighting conditions. However, as disclosed in U.S. Pat. No. 6,013,122, tattoos may include fluorescent or phosphorescent pigments that are normally substantially invisible, but that emit light after exposure to ultraviolet (UV) radiation. The fluorescent or phosphorescent materials down-convert the frequency of the UV electromagnetic radiation to visible colors, because fluorescence and phosphorescence are emissions of electromagnetic radiation at a longer wavelength (lower frequency) than that of the excitation source.

Particles that remain in the dermis typically form a tattoo by affecting the optical properties of the skin. Skin color (brightness, hue, and saturation) is caused by a combination of scattering and absorption of light. Most of the visible light returning from the skin consists of multiply-scattered photons, which have been scattered from dermal collagen fibers (R. R. Anderson et al., “Optics of Human Skin,” J. Invest. Dermatol. 1981; 77: 13-19). Reflectance from the skin surface, which comprises an absolute reflectance of typically only 4-7%, is sensitive to the angle of incidence and the viewing angle. This small component of skin reflectance accounts almost entirely for our view of skin surface texture, “glare,” “oiliness,” etc. (R. R. Anderson, “Polarized Light Examination and Photography of the Skin,” Arch. Dermatol. 1991; 127: 1000-1005). By contrast, most of the visible light reflected from skin is multiply-scattered from the dermis, and this component is Lambertian, i.e., nearly perfectly diffuse. Lambertian reflectors exhibit an absolute reflectance proportional to the cosine of the angle of incidence, which provides some contour cues.

Known tattoos affect skin optics by absorbing certain wavelength bands of visible light, which in turn reduces skin reflectance at these wavelengths, affecting skin color.

### SUMMARY OF THE INVENTION

The present invention provides variable appearance tissue markings with frequency up-converting, condition-dependent appearance and/or retro-reflective properties. The tissue markings according to the present invention can be permanent or those that are designed in advance to be removable. Also, the tissue markings according to the present invention can degrade over time, either naturally or in a predetermined manner.

In certain embodiments, the variable appearance markings of the present invention may become visible or otherwise change appearance only under certain conditions. The material for these markings can be selected such that a the marking blends with the skin and becomes visible or changes appearance only in response to certain stimuli. For example, frequency up-converting tissue markings may stop emitting visible light when the stimulating radiation is discontinued.

There are substantial benefits associated with various kinds of variable appearance tissue markings. Frequency up-converting tissue markings, as opposed to, for example, frequency down-converting fluorescent or phosphorescent markings, require a bright optical source emitting specific wavelengths, e.g., a near-infrared laser or a near-infrared light-emitting-diode (LED), to be seen or “read.” Frequency up-converting tissue markings utilize two or more photons of infrared radiation to excite the emission of one shorter wavelength photon. The intensity of frequency up-converted emission is therefore typically proportional to the square or higher power of the intensity of the infrared excitation. This behavior ensures that low-intensity sources of infrared light, such as most environmental sources, do not excite strong up-converted emission.

In contrast, fluorescent and phosphorescent emission is stimulated by a one-photon excitation process. Fluorescent and phosphorescent emission intensity is generally proportional to the intensity of the excitation. Fluorescent and phosphorescent markings may be simply seen or read by excitation of the fluorescent or phosphorescent emission using ultraviolet light. Thus, frequency up-converting markings may be desirably used for selective identification or fashion.

Condition-dependent appearance tissue markings exhibiting, for example, metachromic properties, can be made to reversibly darken or to change color in response to the aggregation of different dyes.

The invention provides variable appearance markings (such as tattoos) in tissue (typically living tissue, such as skin). These markings are selected and/or designed in advance to be changed and, if desired, removed on demand. These markings are created using indispersible particles that consist of or contain variable appearance materials. The markings may contain particles, which themselves have variable appearance properties. The particles for variable appearance tissue markings may be designed in advance with one or more specific properties (such as electromagnetic and/or structural properties) that allow the appearance of the particles to be altered by exposure to a specific energy (such as a specific electromagnetic radiation), to change and/or remove the tissue markings.

In general, the invention features methods, particles and inks for making variable appearance tissue markings that change appearance upon exposure to certain conditions. Variable appearance markings according to the present invention are those having frequency up-converting, condition-dependent appearance and/or retro-reflective properties. Frequency down-converting fluorescent and phosphorescent tattoo inks and particles, e.g., as described in U.S. Pat. No. 6,013,122 to Klitzman et al. and in U.S. patent application Ser. No. 09/197,105, and photochromic tattoo inks and particles, e.g., as described in U.S. Pat. No. 6,470,891 B2 to Carroll, however, are not considered to be variable appearance tissue markings of the present invention.

Frequency up-converting tissue markings emit electromagnetic radiation at a higher frequency than the excitation frequency. Condition-dependent appearance tissue markings are those that vary in appearance upon a change in their oxidation state or via metachromasia. Retro-reflective tissue markings reflect a portion of the incident light along a path directly backwards to the illumination source.

Certain tissue markings according to the present invention can be removed, on demand, by obtaining particles each including a variable appearance material and designed in advance to enable the particles to be altered, causing either emission of light or decomposition, when exposed to a specific energy (for example, electromagnetic radiation, such as near-infrared (near-IR), infrared (IR), near-ultra violet (near-UV), or high intensity visible radiation).

In certain embodiments, the particles each include indispersible, biologically inert sub-particles, which contain a variable appearance material. The invention also features methods for their formation, application, alteration, and removal.

In some embodiments, the variable appearance particles can be made of a material with a frequency up-converting or a condition-dependent appearance material. In some embodiments, the variable appearance particles can be made of a material with a physical shape, size, and refractive index sufficient for the particles to exhibit retro-reflection.

Particles for use in variable appearance tissue markings that exhibit retro-reflective properties can be, for example, of a spherical, corner cube, cubic crystal or cubic crystal fragment shape. For a spherical particle, the variable appearance material preferably has a refractive index greater than about 1.6, more preferably from about 1.6 to about 2.4. This material may be indispersible. The spherical particles can be of any implantable size larger than about the wavelength of light in the tissue into which the sphere is implanted, and are preferably less than or equal to about ten times this wavelength.

Spherical particles can be formed by, for example, melting selected material, placing the molten material on a disc and rapidly spinning the disc (that is by centrifugal dispersion). The spheres may be sorted by, for example, centrifugation or filtration.

The invention includes methods for making particles for use in a variable appearance tissue marking. The methods can include colliding aerosolized droplets or particles of a core and a coating material; and hardening the coating material, wherein the core comprises a variable appearance material. Alternatively, the methods can include atomizing into a vacuum, gas or liquid an emulsion of a core material in a coating material; and hardening the coating material, wherein the core material comprises a variable appearance material. In another embodiment, the methods include depositing a coating material in a gas or plasma phase onto a solid core particle comprising a variable appearance material to form a solid shell. Other methods include forming a microcapsule of a coating around a core via polymerization or separation by preparing a mixture comprising a core material and a coating material in the same or different emulsion phases; and separating microcapsules, wherein the core comprises a variable appearance material. The methods can also include providing a material having a refractive index greater than about 1.6, e.g., from about 1.6 to about 2.4; and forming a sphere comprising the material with a diameter of greater than about a wavelength of light, e.g., from about one to about ten times the wavelength, in a tissue to be marked so that the variable appearance tissue marking is retro-reflective.

In some embodiments, the sphere is formed by centrifugal dispersion, e.g., by melting the material; placing the molten material on a disc; and spinning the disc. The method can include sorting formed spheres, e.g., by centrifugation or filtration.

In certain other embodiments, the particles each include (i) a coating, which is preferably indispersible, insoluble and/or substantially biologically inert, (ii) a core enveloped within the coating, wherein the core includes the variable appearance material, which is preferably detectable through the coating under certain conditions and is dispersible in the tissue upon release from the particle, and, optionally (iii) an absorption component that absorbs a specific energy and that is located in the coating and/or the core. The absorption of the specific energy can, e.g., rupture the particle, releasing the variable appearance material, which disperses in the tissue, thereby changing or removing, or both, the detectable variable appearance tissue marking.

For example, the dispersible material can be dissolved or metabolized when released into the tissue, or the material can be insoluble and have a size and configuration such that it is physically relocated from the marking by biological processes when released into the tissue. Such dispersible variable appearance materials may be soluble chromophores or dyes, for example, methylene blue or phenothiazinium dyes. The phenothiazinium dyes exhibit a color-changing property called metachromasia, which depends on concentration and aggregation of the dyes. Other compounds may also exhibit color shifts, when aggregated in a core. Oxidation-reduction reactions, which may be reversible or irreversible, can also cause changes in appearance. Methylene blue for example, readily undergoes reversible oxidation-reduction, changing in the process from blue to a transparent material.

In another embodiment, the coating, the variable appearance material, or the optional absorption component, or any combination thereof, absorb specific electromagnetic radiation. The coating can be made of or include a metal oxide, silica, glass, fluorocarbon resin, organic polymer, wax, or a combination thereof. The coating can be substantially visibly transparent and absorb near-IR radiation, for example, at a wavelength between about 750 nm and about 2000 nm. The absorption component can be or include a colored filter glass, graphite, carbon, a metal oxide, an acrylate polymer, or a urethane polymer. The coating can itself absorb, or include an absorption component that absorbs, near-IR, IR, near-UV, or high intensity visible radiation.

In another embodiment, the coating can include pores of a size sufficient to allow the dispersible variable appearance material to leach out of the particle, for example, over a period of weeks or months, so that the tissue marking will no longer be detectible after a given time. In one embodiment, such tissue markings that fade slowly after particle implantation, can also be removed at once upon exposure to the specific energy. The particles can also include multiple cores enveloped within one coating. The invention also features methods for their formation and use.

In another embodiment, the particle includes (i) a coating, which is preferably indispersible and/or substantially biologically inert, (ii) a core enveloped within the coating, wherein the core includes the variable appearance material, and optionally (iii) an absorption component that absorbs the specific energy and that is located in the coating or the core, or both. The variable appearance material may be selected such that its variable appearance properties are altered upon exposure of the particle to the specific energy, thereby changing or removing and/or the marking. The invention also features a method for the particles' formation and use. In this embodiment, the variable appearance materials need not be dispersible, and the particles are not necessarily ruptured.

For example, the particle can be altered by losing its variable appearance properties upon exposure to the specific energy. The particle can further include a sub-particle that has a neutralizing agent that is released from the sub-particle upon exposure of the particle to the specific energy, thereby changing the variable appearance properties of the marking. This agent can be, for example, a chemical bleaching (oxidizing) agent. The variable appearance material can be pH-sensitive, and the agent is an acid, a base, or a buffer capable of effecting a pH transition within the core that changes the material and removes the tissue marking.

The variable appearance material can also be thermolabile, and exposure of the particle to the specific energy heats and alters the material so that variable appearance properties of the tissue marking are eliminated. In this method, the absorption component can be a colored filter glass, graphite, carbon, a metal oxide, an acrylate polymer, or a urethane polymer.

The specific energy can be applied at a wavelength, at an intensity, or for a duration, or any combination thereof, insufficient to completely remove or change the marking, thereby partially removing and/or changing the marking. The specific energy can be applied to effect the rupture or alteration. One feature of the invention is that a single application of the specific energy is intended to be sufficient to effect the rupture or alteration. However, multiple energy applications may also be used.

In another aspect, the invention features a method of changing and/or removing a variable appearance marking created by implanting into tissue a particle comprising a variable appearance material and having a specific property that is designed in advance to enable the particles to be altered when exposed to a specific energy, by exposing the marking to the specific energy for a time sufficient to alter the particles, thereby changing and/or removing the variable appearance tissue marking. In this method, the particles are altered to become substantially undetectable, thereby removing the tissue marking, for example, by rupturing and releasing the variable appearance material, or by eliminating the variable appearance properties of the material.

The coating can provide from about 10 to about 95 percent of the volume (such as 15 to 25 or 35 percent for a coating designed to be ruptured and 40 or 50 to 80 or 90 percent for a coating not designed to be ruptured).

The particles according to the present invention can have an overall size from 50 nanometers to 100 microns. The coating can be or include a metal oxide, silica, glass, fluorocarbon resin, organic polymer, wax, or any combination thereof. In certain embodiments of the rupturable particles, the absorption component forms a plug sealing a hole in the coating, wherein the plug is destroyed upon exposure to the specific energy to open the hole in the coating. Alternatively, the coating can include one or more absorption components, which, when exposed to the specific energy, cause the coating to break open. The variable appearance particles can be sterilized. As used herein, a “particle” is of a size that can be implanted to form a tissue markings. Thus, a particle can be less than 50 nm to 100 μm or greater. In contrast, a “nanoparticle” is specifically a particle in the nanometer (10⁻⁹) size range, for example, 15 nm or 500 nm. A particle or nanoparticle may be of composite construction and is not necessarily a pure substance. It may be spherical or any other shape.

The variable appearance particles (preferably the particles with a non-rupturing outer coating) can further include a sub-particle (that can have its own coating), for example, in the core, that includes a neutralizing agent that is released from the sub-particle upon exposure of the particle to the specific energy, thereby neutralizing the variable appearance properties of the marking. The variable appearance material can be photobleachable, and exposure of the particle to the specific energy renders the marking substantially undetectable. The variable appearance material can also be thermolabile, and exposure of the particle to the specific energy heats and alters the variable appearance properties of the tissue marking. In this marking, the absorption component can be colored filter glass (e.g., made by Schott, Inc., or Dow Corning, Inc., etc.), graphite, carbon, a metal oxide, an acrylate polymer, a urethane polymer, silicon, germanium, metals, organo-metallic crystals, semiconductor materials, etc.

In some embodiments, the variable appearance particles include photobleachable materials, and exposure of the particle to electromagnetic energy renders the particle substantially undetectable. The photobleachable material can be a material whose ability to absorb light can be irreversibly impaired. In some embodiments, the variable appearance material is a multi-photon photobleachable material, e.g., a two-photon photobleachable material, such as a benzophenone, a ketone or a radical generator. In some embodiments, the material is photobleached upon exposure to electromagnetic radiation below about 300 nm.

The invention also features tissue marking inks that include the variable appearance material particles and a liquid carrier, which can include alcohol, water, or glycerin, or any combination thereof.

Additional embodiments are possible wherein the particles do not necessarily include a coating or encapsulation, and the particles are designed in advance with strong absorption of specific energy, which renders the variable appearance material dispersible from the particles or undetectable.

The variable appearance particles of the present invention can be used to mark a variety of tissues including skin, iris, sclera, dentin, muscles, tendons, fingernails, toenails, tissue beneath fingernails, tissue beneath toenails, tissue inside the mouth, and tissue lining internal body passages.

As used herein, a “dispersible” substance (such as a variable appearance material) is (1) dissolved by (and is soluble in) bodily fluids, for example, those within a cell or tissue; (2) metabolized (including digested) by living tissue and/or cells into one or more new chemical products; and/or (3) of a size (on average no larger than about 50 nm, but in some cases necessarily much smaller, for example, less than about 5 nm), made of a material, and configured such that normal bodily processes result in its physical relocation from tissue (from cells or from extracellular matrix).

As used herein, an “indispersible” substance (such as a coating material or an individual particle) does not disintegrate, dissolve, or become metabolized in tissue. “Indispersible” particles are also large enough on average (generally greater than about 50 nm, but depending on the material as small as 5 nm or even smaller) and have a configuration on average such that when a plurality is implanted into tissue, a sufficient number is retained to form a detectable marking, even though some number of the individual particles may be relocated from the tissue marking site through biological processes (such as lymphatic transport).

An “inert” or “biologically inert” substance (such as the coating material of a particle) generally creates no significant biochemical, allergic, or immune response after the normal healing period when implanted into living tissue.

A “variable appearance material” is broadly defined herein as a substance (solid, liquid, or gas) that has frequency up-converting, condition-dependent appearance, or retro-reflective properties. Condition-dependent variable appearance properties include metachromasia and the ability to change color based on the oxidation state. One or more of these properties impart a variable appearance to tissue markings under different illuminations, excitations, or conditions. Different illuminations can include, for example, illumination with a substantially collimated light source rather than a substantially diffuse light source. For example, a frequency up-converting material may normally be substantially invisible, but can emit visible light after exposure to infrared radiation. A condition dependent material may be, for example, a metachromic material. A retro-reflective material may be, for example, any glass that has a sufficient refractive index so that particles such as spheres of the glass within the tissue marking retro-reflects incident light.

“Color” is broadly defined herein as a detectable (that is, visible or able to be made visible under certain lighting conditions, or able to be detected using a detecting device for electromagnetic radiation outside the visible spectrum, for example, an infrared camera) property determined by a substance's electromagnetic absorption and/or emission spectrum (that is, in the ultraviolet, near-ultraviolet, visible, near-infrared, infrared, and other ranges). Black and white are colors under this definition.

As used herein, a substance (such as a variable appearance material) is “invisible” when substantially no color can be detected (such as in a tissue marking site) apart from the normal coloration of the substance's surroundings (such as skin or other tissue) by the naked eye under normal lighting conditions, for example, diffuse sunlight or standard artificial lighting. A substance is “undetectable” when it is invisible to the naked eye under normal lighting conditions, and also invisible by the naked eye, or a device, under any other lighting conditions (such as fluorescent, UV, or near-infrared).

As used herein, a “permanent tissue marking” or “tissue marking” is any mark created by the introduction of particles of the invention into tissue, typically living tissue, with the intention of permanent or long-term endurance. Markings can be any color and must be detectable, for example, by the naked eye or by a detection device. A permanent marking is generally a marking that remains visible or otherwise detectable until it is exposed to a specific energy. However, in certain embodiments, a permanent marking can be a mark that is designed in advance to disappear after a predetermined time, for example after one or several months, and/or can be removed by exposure to a specific energy before the predetermined time.

As used herein, the terms “pH-sensitive,” “thermolabile,” and “photobleachable” refer to markings whose appearance altering ability is affected by exposure to a certain pH, temperature, and electromagnetic radiation respectively. As used herein, these terms are not synonymous with pH-varying, thermochromic and photochromic.

For example, a metachromic tissue marking, which can vary in appearance from red to blue but which can no longer change its color from red to blue via metachromasia upon being exposed to a certain pH, is considered a “pH-sensitive” tissue marking of the present invention. Exposure of such a tissue marking to that certain pH destroys its metachromic ability. Preferably, if it is desired to remove the tissue marking, the conditions that destroy the ability of the tissue marking to vary in appearance also render the tissue marking invisible or, more preferably, undetectable. For example, while the above-mentioned pH-sensitive metachromic tissue marking can permanently remain either blue or red upon exposure to a certain pH and no longer respond to metachromic conditions, it is preferable that the pH change make the tissue marking either invisible or undetectable.

Photobleachable, pH-sensitive, and thermolabile properties are related solely to the issue of removing the variable tissue markings and/or destroying the tissue markings' variable appearance characteristics and not merely to a change from a certain appearance to being substantially undetectable. It should be noted that the photobleachable, pH-sensitive, and thermolabile tissue markings according to the present invention are different from neutralizable, pH-sensitive, and thermolabile markings disclosed in U.S. patent application Ser. No. 09/197,105, because this document does not disclose or suggest removing tissue markings that are metachromic, frequency up-converting, retro-reflective, or tissue markings that vary in appearance based on their oxidation state.

As used herein, a “tattoo” is a type of tissue marking wherein the tissue is usually skin. “Standard tattoos” and the pigments used to create them have not been designed in advance for appearance change and/or removal.

As used herein, a “non-invasive” procedure for creating a tissue marking implants particles into the tissue without the use of an implement that enters the surface of the tissue. Forces that can be applied to particles to achieve non-invasive tattooing include ballistic, electrical (such as through iontophoresis or electroporation), magnetic, electromagnetic, ultrasonic, chemical, and chemical gradient forces, or any combination of these forces.

As used herein, “removal” of a tissue marking means either the physical removal of the substance(s) that create the appearance of the marking, or the destruction or facilitated loss of some variable appearance property that renders the marking substantially undetectable. Thus, all, some, or none of the components (variable appearance material, coating material, etc.) of the particles may be physically relocated from the tissue when a tissue marking is “removed.”

Tissue marking particles that are “designed in advance” for change and/or removal means that the materials and/or structure of the particles are selected and/or engineered, and intended, to facilitate change and/or removal of the tissue marking. It in no way implies that a pre-determined removal method must be used, that this or another removal method is the best method, or that a removal method is explicitly outlined at the time of particle design. Multiple removal methods may be acceptable for removing a given marking. Adjustments made to any proposed method may affect removal efficacy positively, negatively, or not at all.

Conventional permanent black skin tattoos are used to mark regions of the body treated during radiation therapy for cancer. These tissue markings are often an unsightly reminder of having had cancer. Tissue markings with retro-reflective properties described in this invention can be used for alignment during radiation therapy, but remain invisible under normal lighting conditions.

Tissue markings are used for a number of reasons. These reasons include body art, a rite of passage, spiritual outlet, emotional transformation and ritualistic identification. The latter is particularly pertinent to gang tattoos, which identify an individual with a certain street gang. Tissue markings have been employed as a sign of rebellion against parents or society (e.g., a jailhouse tattoo). Sometimes, a tissue marking is obtained in memory of a loved one or to show affection, such as tattooing a significant other's name.

In many cases, however, people simply want to have what they consider to be a distinctive or fashionable tattoo. Variable appearance tissue markings of the present invention can be used to achieve this objective.

In addition, variable appearance tissue markings can be used, for example, as normally-invisible, or encoded, identification and/or information markings on skin (for “reading” by, e.g., military or medical personnel), as identification markings on the skin or within the body for reference by doctors during or following medical treatment, as cosmetic or artistic markings on skin (tattoos, permanent makeup, and suntans), as identification markings on pets, as diagnostic markings (to indicate presence of disease or exposure to certain conditions, such as electromagnetic radiation of a certain frequency), etc.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

The tissue markings of the present invention provide several advantages over known tissue markings, e.g., a variable appearance as defined herein and the ability to remove the same.

Other features and advantages of the invention are apparent from the following detailed description and from the claims.

### BRIEF DESCRIPTION OF THE DRAWINGS

FIG. 1 is a schematic cross-sectional view of a variable appearance particle.

FIG. 2 is a schematic cross-sectional view of a particle containing variable appearance nanoparticles.

FIG. 3 is a schematic cross-sectional view of a particle containing sub-particles comprising encapsulated variable appearance material.

FIG. 4 is a schematic cross-sectional view of a particle containing a neutralizable variable appearance material and a sub-particle comprising an encapsulated neutralizing agent.

FIG. 5 is a schematic cross-sectional view of another embodiment of a variable appearance particle.

FIG. 6 is a schematic cross-sectional view of a particle containing a variable appearance material.

FIG. 7 is a schematic illustration of retro-reflection in a sphere.

FIG. 8 is a schematic illustration of a set-up used to measure retro-reflective strength of a tissue marking.

### DETAILED DESCRIPTION

Variable appearance tissue markings of the present invention are made of particles that are capable of changing their appearance, and thus the appearance of the tissue marking, upon exposure to specific conditions. One type of a variable appearance tissue marking is a tissue marking that has frequency up-converting properties, which allow the marking, for example, to emit visible light when it is exposed to near-IR light. Another type of a variable appearance marking is a marking that can vary in color depending on the environmental conditions to which it is exposed. Yet another type of a variable appearance tissue marking is a marking that can retro-reflect incident light.

The particles of the present invention preferably meet several conditions in addition to the optical and other properties described. First, the particles are preferably indispersible, as described herein, in the tissue under normal physiological conditions. Second, any component of the particles that will at any time (such as during implantation or removal or while the marking exists) come into contact with the tissue is preferably substantially biologically inert, unreactive, or safely metabolized.

It is theoretically possible to select a palette of pure materials that meet both of the above criteria for use as tissue marking particles. Since some variable appearance materials may not meet either of these criteria, a more efficient way to design the particles is to prepare them as composites of two or more materials. The combination of several materials' properties can more easily satisfy the two criteria. For example, the variable appearance material may satisfy criteria 2 and a coating may satisfy criteria 1 and 2.

Particles

Particles of the invention are preferably substantially biologically inert. These particles may have a coating material enveloping at least one core comprising one or more variable appearance materials. The particles typically have a diameter from about 50 nm to about 100 microns, but may be smaller or larger as long as the particles can be implanted into a tissue to provide a tissue marking. They can be spherical, as shown in the figures, or any other shape, so long as these markings maintain their variable appearance properties.

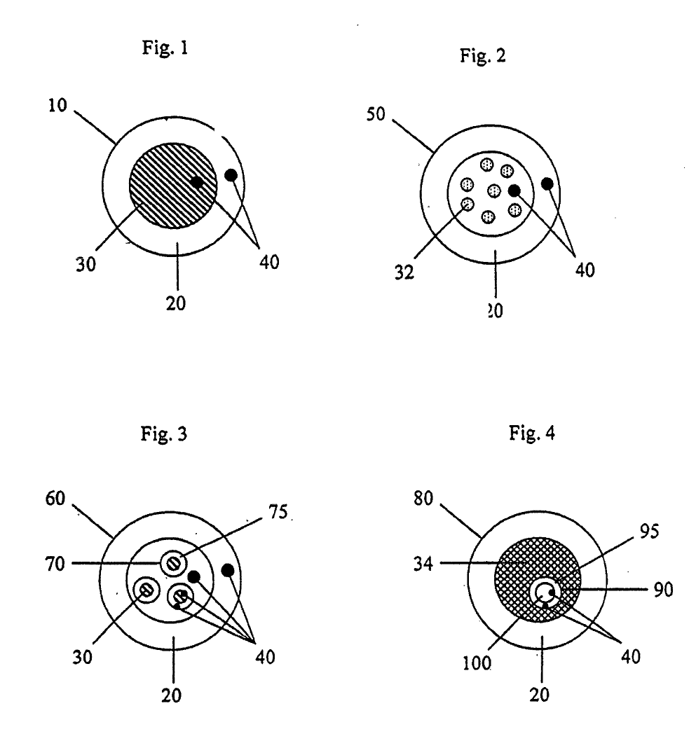

FIG. 1 shows a basic particle 10, which includes a coating 20 encapsulating a core containing variable appearance material(s) 30. As shown in particle 50 of FIG. 2, the core may contain discrete variable appearance nanoparticles 32.

In certain cases, as depicted in FIG. 3, it may be useful to encapsulate a plurality of composite sub-particles 70, comprising variable appearance material(s) 30 and substantially transparent coating 75 (which may or may not be the same material as used in coating 20), in coating 20 to form particle 60. Sub-particles 70 can be any size as long as they fit within the particle 60.

In another embodiment, illustrated schematically in FIG. 4, neutralizable variable appearance material(s) 34 and composite sub-particle(s) 90 (comprising neutralizing agent(s) 100 and coating 95) are encapsulated in coating 20 to form particle 80.

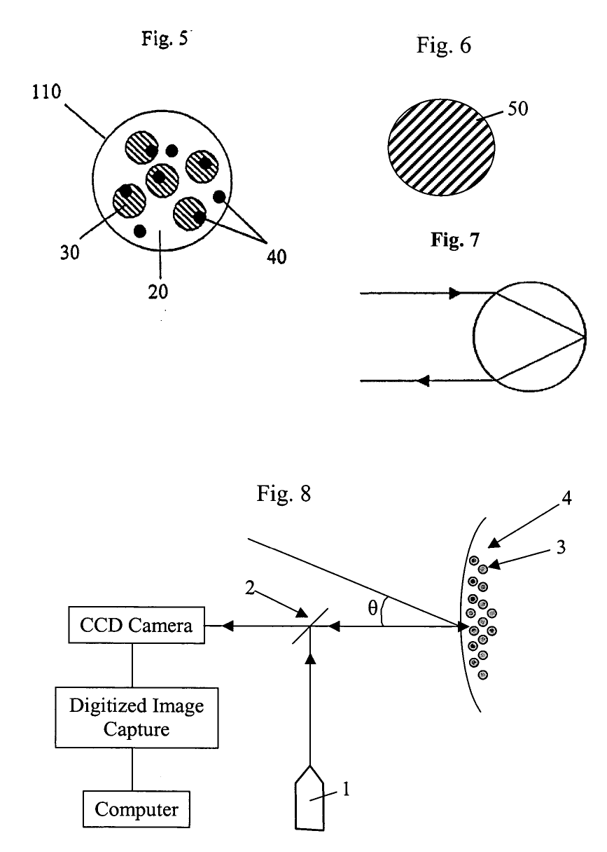

FIG. 5 depicts an optional configuration for the particle in FIG. 1, where two or more cores containing variable appearance material(s) 30 can be present within the coating 20 of a single particle 110. Analogous multi-core versions of the particles in FIGS. 2 to 4 can also be constructed.

Generally, coating 20 and/or 75 or 95 is made from any substantially transparent material(s) (that is, a material that allows the encapsulated variable appearance material to be detected, for example, seen) that is indispersible (and is therefore generally retained in tissue) and is biologically inert under physiological conditions. The coating can have a thickness ranging from about 0.05 r (about 86% core loading, 14% coating, by volume) to about 0.6 r (about 6.4% core loading, 93.6% coating, by volume), where r is the particle radius. The coating can be from about 10 to about 95 percent of the total volume of a particle.

Any substance or combination of substances that imparts variable appearance characteristics to a particle and which is usually, but not necessarily in all cases, inert and unreactive in the body, may be chosen as variable appearance material(s) 30, 32, or 34. This substance can be subject to removal (or alteration) according to one of the two general methods described in detail hereafter, or another suitable method.

Depending on the planned removal method of the particles depicted in FIGS. 1 to 5, an additional absorption component(s) 40 may or may not be incorporated into coatings 20, 75, or 95, and/or mixed with variable appearance material(s) 30, nanoparticles of variable appearance material(s) 32, or neutralizing agent(s) 100.

The particles schematically depicted and described generally herein can be constructed in two embodiments according to the intended removal method (except for particle 80, which is specific to a single removal method). In the first embodiment, particles can be constructed to contain dispersible variable appearance materials that are removed when particles are made permeable, for example, by rupture of a coating. In the second embodiment, particles can contain variable appearance materials that are rendered invisible without rupturing the particles.

More specifically, according to the first embodiment, particles 10, 50, 60, and 110 can contain dispersible variable appearance material(s) 30 or 32. Tissue markings made using these particles can be removed when desired using a method wherein the tissue marking is exposed to specific electromagnetic radiation, which ruptures the particles. For example, the particles can rupture as the result of heating, for example, when the coating 20 and/or 75, variable appearance material(s) 30 or 32, or additional absorption component(s) 40 absorb the specific radiation. In this embodiment, when the variable appearance materials are dispersed from the tissue marking site, the tissue marking disappears. This can occur over the course of several minutes to several weeks following irradiation.

Particles 10, 50, and 110, which contain dispersible variable appearance material(s) 30 and 32, can also be constructed with porous coatings such that the variable appearance material(s) leaches out and is dispersed over time. If desired, these particles can also be designed in advance for removal or rendering them invisible using specific electromagnetic radiation as in the above description.

According to the second embodiment, particles 10, 50, 60, 80, and 110 can contain specific encapsulated variable appearance material(s) 30, 32, or 34, whose variable appearance properties can be changed upon exposure to a specific type of electromagnetic radiation so that the marking becomes undetectable. For example, one such type of electromagnetic radiation is in the form of pulses from a laser operating at a specific wavelength. In particles 10, 50, 60, or 110, this radiation should cause variable appearance material(s) 30 or 32 to be neutralized, oxidized, reduced, thermally altered, or otherwise destroyed, usually through absorption of the radiation by the variable appearance material(s). Additional absorption component 40 is usually absent from the coating material in this embodiment.

In particle 80, this radiation must cause neutralizing agent 100 to come into contact with neutralizable variable appearance material(s) 30, usually through rupture of sub-particle(s) 90 via absorption of radiation by coating 95, neutralizing agent 100, or additional absorption component(s) 40. Alternatively, neutralizing agent 100 may be an activated bleaching agent, e.g., a photothermally or thermally triggered free radical generator. Activation of the neutralizing agent 100 by externally-applied energy can cause chemical reactions, which alter or remove the appearance of the tissue marking.

The variable appearance properties of a tissue marking made using these particles 10, 50, 60, 80, and 110 can be eliminated when desired using a method wherein the tissue marking is exposed to the specific electromagnetic radiation described above. In this embodiment, particles are not necessarily ruptured, and the variable appearance materials are not necessarily released into the bodily fluids, but the particles become undetectable. Thus, the tissue marking is removed during or after irradiation, usually within milliseconds to minutes, although none of the components of the particles are necessarily physically relocated from the tissue.

The variable appearance tissue markings need not consist of encapsulated materials. As shown in FIG. 6, these markings can consist of unencapsulated particles made of a variable appearance material 50. This material is preferably substantially biologically inert and indispersible in tissue. The unencapsulated form is particularly preferred for tissue markings having retro-reflective properties, which are described below in detail. Such variable appearance markings may be removed by conventional laser treatment, and they may also be designed in advance such that they could be easily destroyed.

Certain aspects of the design of the several particles described herein may be interchanged or omitted, yielding useful particles. These and other types of particles are within the scope of the invention and will be useful if they are in the size range capable of providing tissue markings.

Coating Materials

The material(s) for coating 20 should preferably be indispersible and substantially biologically inert and substantially visibly transparent. Substances fitting these criteria that are capable of encapsulating variable appearance materials useful in the invention include waxes with a melting point substantially above body temperature, for example, natural waxes, synthetic waxes, and mixtures, specifically Polywax™ and carnauba wax; plastics and organic polymers, for example, parylenes, polyamide, polyimide, polyvinyl acetate, urea formaldehyde, melamine formaldehyde, ethylene acrylate, cyanoacrylates, polymethyl-methacrylate, butadiene-styrene, and specifically biocompatible materials such as Epo-Tek™ 301 and 301-2, manufactured by Epoxy Technology, Billerica, Mass.; metal oxides, for example, TiO₂, silica (SiO₂), BIOGLASS®, KG-3 and BG-7 manufactured by Schott, Inc., Germany, and other glasses (SiO₂ plus any one or more of the following: Na₂O, CuO, B₂O₃, MgO, Al₂O₃P₂O₅, and others); inorganic fluorine-containing compounds such as MgF₂; and fluorocarbons such as TEFLON®.

In some embodiments, coating 20 is made of a material or includes specific absorption component(s) 40 that strongly absorbs in a particular spectral region, for example, ultraviolet, visible, infrared (such as part of the near-infrared from 800 to 1800 nm), microwave, or radio wave. The choice of such a material allows particles to be selectively heated and ruptured by radiation (such as from a laser) near the absorption maximum of said material, thereby releasing dispersible variable appearance materials. For reasons of avoiding electromagnetic radiation absorption by surrounding tissue during removal treatment, the spectral region from about 800 nm to 1800 nm is most desirable, particularly for condition-dependent appearance particles, as described in more detail in the Removal Methods section.

The entire coating 20 can be made of an absorbing material allowing rupture through absorption of specific electromagnetic radiation, for example, by differential heating, which fractures the coating, or indirect heating of the central core and rapid expansion, which explodes the coating.

Other useful variations of this embodiment include making a small portion of coating 20 with an absorbing material or adding specific absorption component(s) 40. Irradiation then selectively affects the absorbing portion of the coating, causing the particle to rupture and its contents to be exposed to bodily fluids. The absorption component 40 can act like an “egg tooth” that ruptures the coating or like a “plug” that is destroyed to allow the variable appearance material to escape from the coating.

Examples of useful materials for constructing infrared-absorbing coatings 20 or specific absorption component(s) 40 are Schott filter glasses that absorb certain near-infrared wavelengths and are transparent or nearly transparent in visible light at the thicknesses of the coatings used in the particles. For example, KG-3 filter glass (Schott, Inc.) is designed to absorb infrared light at 1000 to 1400 nm, with a maximum at 1200 nm. BG-7 filter glass (Schott, Inc.) is designed to have an absorption maximum at 800 nm. Other examples of useful infrared- or near-infrared-absorbing materials include graphite and other forms of carbon; metals, metal oxides, and metal salts; and polymers such as acrylate and urethane.

Useful materials for absorption component(s) 40, which absorb in non-infrared regions of the electromagnetic spectrum, include ferrites (such as iron oxides), which strongly absorb short, high intensity pulses of light, near-infrared, microwaves or radio waves. Use of these materials allows the particles to be heated and ruptured when irradiated with microwaves or radio waves of the proper wavelength, intensity, and pulse duration.

Electromagnetic absorbing materials that have a color under visible light can also be used for the coating material if that color is desired, or is eliminated, for example by exposing the material to a specific type of radiation. If used as absorption component(s) 40 (and in some cases even as coating 20), these materials may be effectively invisible because of the small amounts/thicknesses in the particles.

In some instances, it may be desirable to provide a coating 20 for particle 10 that is porous. For example, a porous coating enables variable appearance materials to leach slowly out of particles to provide a tissue marking that lasts for a specific length of time, for example, a few weeks or months. Tissue markings made from such porous particles fade over time until the variable appearance materials have leached out of the particles. The length of time required for the marking to become invisible can be controlled by adjusting the size and number of pores in coating 20. Pores can be introduced into coatings during the encapsulation procedure.

Coatings may also be provided for retro-reflective particles. Such coatings may be colored or transparent. It is preferable that the coating be much thinner than a wavelength of light, e.g., less than about 100 nm thick. In this case, the coating's refractive index is nearly irrelevant and the retro-reflective properties of the tissue marking are controlled by the shape, size and the refractive index of the material used to construct the particle.

Frequency Up-Converting Materials

A frequency up-converting material is a material, which, when used in a tissue marking of the present invention, detectably radiates at a shorter wavelength than the wavelength of the exciting radiation. Primarily, the exciting radiation for a single frequency up-converting compound is in a narrow wavelength band, for example, near-IR. One such frequency up-converting material is sodium yttrium fluoride.

Frequency up-converting materials are available over a very wide optical spectrum, from short-wavelength (high frequency) ultraviolet to long-wavelength (low frequency) infrared. The materials preferably used in the variable appearance tissue markings of the present invention are materials that emit in the wavelength range from about 350 nm-1300 nm. These wavelengths are able to easily “get out” from the dermis. A number of up-converting materials are discussed in Phosphor Handbook (S. Shionoya and W. M. Yen, editors) CRC Press, 2000. pp 643-650.

Preferably, frequency up-converting materials are biologically inert and/or non-toxic (ideally they are non-carcinogenic, non-allergenic, and non-immunogenic), such as those approved by the FDA for use within the body. However, they need not necessarily be known to be non-toxic in those embodiments in which the coating is impervious to bodily fluids and is maintained intact, even during removal and alteration.

Frequency up-converting materials may be mixed in combinations for various purposes before or after encapsulation, so that a variety of appearances and emissions can be obtained when excited by a variety of excitation wavelengths. Markings that are invisible under normal lighting conditions can be encoded using materials with different up-converting excitation and/or emission wavelengths. For example, different frequency up-converting materials can be implanted into tissue separately or together, and may be encapsulated separately to result in desired multi-wavelength emissions. Combinations of two or more frequency up-converting materials can be mixed and then encapsulated to form particles.

Optionally, as illustrated schematically in FIG. 3, a particular frequency up-converting material 30 may be separately encapsulated to form sub-particles 70, and then sub-particles containing different materials can be mixed together (or with unencapsulated frequency up-converting material). The mixture can then be encapsulated in coating 20 to form a particle having a measured or perceived emission(s) resulting from the blend of different frequency up-converting materials. Also, frequency up-converting materials can be combined with condition-dependent appearance materials.

The particles may be constructed so that they are composed, essentially, of the frequency up-converting material, or the particles contain such material in a quantity sufficient to produce detectable frequency up-converting effects. These particles need not encapsulate the frequency up-converting material, but may simply consist partially or entirely thereof.

Frequency up-converting particles are sometimes used as biochemical probes for detecting proteins or pathogens, and are commercially available for this and similar purposes (for example, TAL Materials Inc. produces mixed-metal up-converting phosphor nanoparticles) when attached to carrier molecules such as antibodies. Commercially available frequency up-converting particles may be biologically toxic unless encapsulated as described. Efficient frequency up-converting materials have been described, such as erbium yttrium niobate, YNbO4:Er3+ (for example, J Silver, P J Marsh, R Withrall “Efficient Upconversion Luminescence from YNbO4:Er3+,” Proc. First International Conf. on Science and Technology of Emissive Displays and Lighting, p. 147-150, 1999), erbium yttrium fluoride, and others. Frequency up-converting processes include simultaneous two-photon, sequential two-photon, and photon avalanche excitations. Most materials with high up-converting luminescence efficiency are composed of glasses or crystals with various transition metals. A wide range of infrared and visible excitation frequencies, and emissions in the UV, visible and near-infrared spectrum have been described, some of which are used for frequency conversion in lasers. However, none of these frequency up-converting materials or processes have been configured, used, or reported as tissue markings. When used as a tissue marking, the emission may be either visible light, or for invisible emission preferably in the near-infrared spectrum.

For example, the inventors have verified that erbium (10%) yttrium (40%) fluoride nanocrystals can be used to make a frequency up-converting tissue marking in a rabbit. The frequency up-converting material is excited at 980 nm and emits green light.

Materials with Condition-Dependent Appearance

Materials with condition-dependent appearance are used in particles for tissue markings that can be changed between/among two or more appearances. Specifically, these materials change their appearance via being oxidized and/or reduced and/or via metachromisia.

Substances that alter their appearance with oxidation or reduction (electron exchange) are, for example, dyes that are reversibly oxidized or reduced, such as methylene blue. In addition, sunbstances such as transition-metal oxides, hydroxides, aqueous solutions of their salts and complexes can be used. Transition metals may be, for example, copper, iron, cobolt, manganese, chromium, etc.

Furthermore, dyes used as pH indicators undergo a reduction in a reaction with hydrogen ions or an oxidation in a reaction with hydroxyl ions and can change their appearance. Indicators that change appearance in the visible light range are, for example, methyl violet, crystal violet, ethyl violet, malachite green, methyl green, cresol red, thymol blue, bromophenol blue, congo red, methyl orange, resorcin, alzarin red S, methyl red, bromoceresol purple, chrophenol red, bromothymol blue, phenol red, neutral red, phenolphthalein, thymophthalein and andalzarin yellow R. Flourescent indicators (used as indicators of intracellular pH) are, for example, fluorescin, carboxyfluorescin and derivatives, pyranine, LysoSensor probes, Oregon Green® carboxylic acid and 9-amino-6-Chloro-2-methoxyacridine (see the world wide web at probes.com/handbook/print/2101.html).

Metachromic substances are, for example, phenothiazinium dyes or cyanine dyes. Also, ionic dyes that bind in solution with polyelectrolytes, such as proteins, nucleic acids, or polysaccharides that bear ionized groups on their chains, can be metachromatic variable-appearance materials according to the present invention. In some embodiments, the polyelectrolyte that is bound by the dye is included in the particle with the dye, e.g., encapsulated within the particle with the dye.

For example, toluidine blue is an example of a usable ionic dye. Toluidine blue stains nucleic acids blue (the orthochromatic color), but stains sulfated polysaccharides purple (the metachromatic color). When dye molecules bound to sulfate groups are stacked closely together, the dye experiences a color shift from blue to purple.

Another example of an ionic dye is brilliant cresol blue. This dye blue shifts when bound to glycosoaminoglycans (GAGs) such as heparin and chondrotin 4-sulfate in aqueous solution.

Yet another example of an ionic dye is methyl green pyronine. This dye is red when bound to RNA, but is otherwise green.

Further examples of useful metachromatic materials include fluorescent nucleic acid stains that exhibit enhanced fluorescence or an emission spectra shift when bound to nucleotides. An example of a fluorescent nucleic acid stain is ethidium bromide. A SYTOX® green stain (Molecular Probes, Eugene, Oreg.) exhibits enhanced fluorescence only when bound to DNA. Another example of a fluorescent nucleic stain is acridine orange, which is a dual nucleic acid stain that has a green fluorescence emission maximum at 525 nm when bound to DNA. Its emission maximum is shifted to 650 nm when bound to RNA (see the world wide web at probes.com/handbook/print/0801.html).

Preferably, like frequency up-converting materials, these materials are, or are made to be (e.g., by encapsulation), biologically inert and/or non-toxic (ideally they are non-carcinogenic, non-allergenic, and non-immunogenic), such as those approved by the FDA for use within the body. However, they also need not necessarily be known to be non-toxic in those embodiments in which the coating is impervious to bodily fluids and is maintained intact, even during removal and alteration.

Condition-dependent appearance materials may be mixed in combinations before or after encapsulation, so that it may only be necessary to select a small number of different materials to obtain a broad range of colors for various tissue marking purposes. Selection of a desired color pattern may be achieved, for example, by color mixing according to the three “primary” color principle similar to RGB computer monitors or in the same manner as an artist blends any pigments until a satisfactory blend is achieved.

Some of the above embodiments of the present invention can contain dispersible variable appearance materials. These materials should be (1) water-soluble at physiological pH, although fat-soluble materials will also work if they are rapidly flushed from tissue, or (2) digestible or metabolizable through enzymatic pathways (such as methylene blue, which is rapidly metabolized by mitochondrial reductases, and proteins which are digested by proteases). In some cases, it may be possible to modify the material to improve its dispersibility.

Dispersible variable appearance nanoparticles can be made from certain inert, normally indispersible substances, which have been reduced to nanoparticles about 50 nm and smaller. Although diffuse nanoparticles might have different optical properties from the macroscopic material, when concentrated within the confined space of a particle core (that is, nanoparticles are closer together than the wavelength of visible light, about 500 nm), they produce the same effects as the original indispersible material from which they are derived. In contrast to the macroscopic material, some nanoparticles are poorly retained in tissue and are rapidly dispersed through lymphatic transport as demonstrated in lymphangiography experiments. Useful dispersible nanoparticles may be made from graphite, iron oxides, metal oxides, metal salts, metals, organo-metallic compounds and other materials with small particle size, for example, less than 50 nm and preferably less than 5 nm.

Like the coating material, the core can contain variable appearance material(s) 30 or can also include specific absorption component(s) 40, which strongly absorbs radiation of specific wavelength(s), particularly in the near-infrared spectral region from about 800 to 1800 nm. Absorption properties of the variable appearance material or specific absorption component allow the particle core to be selectively heated, thus rupturing the particle and releasing the previously encapsulated variable appearance material.

Absorbing materials, such as near-infrared absorbing materials, used as specific absorption component(s) 40 can be visibly transparent or nearly transparent at the concentrations and sizes used within the particles so that they do not affect the perceived color of the particle or of the tissue after particle disruption even if the material is indispersible. Useful examples include particles of filter glass (such as those manufactured by Schott, Inc.) and plastics such as polymethylmethacrylate (PMMA), as well as low concentrations of nanoparticulate graphite, carbon or metals such as silver or gold. Silver and gold nanoparticles may be configured to produce plasmon resonance, a quantum effect accounting for very high light absorption at certain wavelengths. These materials can be mixed with desirable frequency up-converting materials and then encapsulated.

Although this description has focused on near-infrared absorbing materials, materials with other properties (such as absorption of ultraviolet, visible, microwave, radio wave and other wavelengths) can also be used to construct the radiation-targeted portion of the particles.

In another embodiment, variable appearance material(s) 30, 32, or 34 can be materials that are rendered undetectable upon exposure of the particles to specific electromagnetic radiation without necessarily rupturing the particle. Neutralizable variable appearance materials (which react with a neutralizing agent released by the radiation), photobleachable variable appearance materials (altered by the radiation) or thermolabile variable appearance materials (altered by heat produced by radiation absorption) may be used. When variable appearance materials have undesirable toxicity, the tissue should not be exposed to them. A coating surrounding such materials can be made to be difficult to rupture, for example, through increased thickness or exceptional pliancy for resilience, so that exposure of the particles to radiation alters the color of the wavelength frequency up-converting materials without rupturing the particles. Although dispersible materials are suitable, these variable appearance materials need not be dispersible because they are not intended to be released.

Neutralizable frequency variable appearance materials can be used in the particles depicted in FIG. 4. Variable appearance materials that are pH-sensitive can also be used, because they can be rendered undetectable if the pH within the particle is changed.

Photobleachable variable appearance materials that are rendered undetectable or invisible by exposure to a specific type, wavelength, and/or intensity of electromagnetic radiation can be used. Some variable appearance materials are only photobleached upon simultaneous absorption of multiple photons, and are therefore unaffected by diffuse solar radiation.

A thermolabile variable appearance material may be any variable appearance substance that becomes undetectable or invisible upon heating through absorption of radiation by the material or a component in contact with the material, which indirectly heats it. Thermolabile variable appearance material mixtures can also be prepared by mixing a variable appearance material with a thermally initiated activator that releases free radicals upon heating. These free radicals then react chemically with the variable appearance material to render it undetectable. The activators are used in the plastics industry for thermal curing of various plastics.

Unencapsulated Particles

The particles may be constructed so that they are composed, essentially, of the variable appearance material, or the particles contain such a material in a quantity sufficient to produce detectable variable appearance effects upon implantation into tissue. Preferably, the materials that make up these particles are non-dispersible and substantially biologically inert.

For example, a non-encapsulated variable appearance particle may comprise a crystalline form of sodium yttrium fluoride or erbium yittrium fluoride nanocrystals. These crystalline forms may be shaped into particles that can be injected into tissue and form a tissue marking, as described below and shown in FIG. 6.

The unencapsulated particles are particularly preferred for retro-reflective and frequency up-converting tissue markings.

Tissue Markings with Retro-Reflective Properties

Retro-reflection is reflectance of incident light along, or nearly along, a direct path toward the illumination source. One of the most perfect retro-reflectors is a corner-cube prism. For example, a corner-cube retro-reflector was placed on the moon's surface by hand, which to this day provides highly accurate measurement of the distance between the earth and the moon, by measuring the time of flight of a light beam reflected exactly back toward its source on earth.

A common example of a corner-cube retro-reflector is a clear plastic automobile tail-light cover. This cover allows light to pass through, for example, from a turn-signal lamp, but strongly retro-reflects incident light from another automobile's headlamps, especially at night. Less perfect retro-reflectors consist of cubic crystal fragments, which tend to act as an ensemble of light scattering particles with high reflectance in the reverse direction compared with other directions. Some retro-reflective sign paints use microscopic cubic crystals for retro-reflectors.

More commonly, glass or other transparent spheres are used for retro-reflectors in night safety paints, clothing, highway markers, etc. This is a preferred shape for retro-reflective tissue markings, because of its simplicity and the ability to be made from a wider variety of materials. Furthermore, due to their symmetry, spheres can act as retroreflectors regardless of the relative orientation between the sphere and the source of light.

Angular scattering from perfect spheres, as a function of refractive index of the sphere and its surrounding medium, and polarization of incident light was described in detail in 1908 by Mie, who expanded Lord Rayleigh's theory of molecular light scattering. Mie's theory is well-known in optical physics. According to this theory, spherical particles with a refractive index greater in a specific relation to the surrounding medium act as strong retro-reflectors under certain conditions.

There are several modes for retro-reflection from spheres. Essentially, the strongest retro-reflection is achieved when incoming light is refracted, according to Snell's law, at the front surface of the sphere to a focus at the rear surface of the sphere, which acts as a partial mirror, returning the light along the path of incidence. This simple mode of retro-reflection is illustrated in FIG. 7. However, according to Mie's theory, there are other modes of retro-reflection that involve multiple internal reflections within the spherical particle and/or optical wave interference in which constructive interference is produced in the direction of retro-reflection.

Knowing the refractive index of the tissue into which the marking particles are to be implanted, it is possible to use Mie's theory to calculate the refractive index and the size of spherical particles made of a transparent material, which will act as strong tissue marking variable appearance retro-reflector particles. One of the basic caveats of this theory is that geometrical (classical ray) optics are a good approximation of optical scattering for particles, which are equal to greater than about 10 times the wavelength of light in the medium. Between about 1 to 10 times the wavelength, a transition occurs in scattering properties from those predicted by geometrical optics and Mie's particle-scattering theory. Accordingly, the preferred size of a retro-reflective particle that can be used in tissue markings is from about 1 about 10 times the wavelength of the light in the medium into which the particle is implanted.

Retro-reflective variable appearance particles can be used over the entire optical spectrum, which penetrates into the tissue. This range is from about 0.321 μm to about 2.0 μm. For visible wavelengths (0.4-0.7 μm), the most desirable size range for particles is about 0.5 μm to about 5.0 μm. Spheres much smaller than 0.5 μm will act as point scatterers, with weak retro-reflection. Spheres much larger than 5.0 μm can be excellent retro-reflectors, but may be larger than the ideal size for tissue markings. However, certain large spheres can be implanted in an extracellular location as described above.

The medium for receiving the variable appearance retro-reflective markings can be, for example, human skin. Since the wavelength of visible light in a vacuum, as mentioned above, is 0.4-0.7 μm (e.g., 0.5 μm) and the refractive index of human skin is approximately 1.35-1.40 (e.g., 1.35), the wavelength of visible light in the dermis is, therefore, for example, about 0.5/1.35=0.37 μm.

Particles up to ten times this wavelength are still small enough to make a good tissue marking, because these sizes fall well within the range used for marking tissue. Therefore, spherical particles for can act as strong retro-reflective tissue markings.

A retro-reflective tissue marking particle need not necessarily be a solid sphere. For example, encapsulated high-index oil or dissolvable core material can be used.

The strength of back-reflected light is very sensitive to particle index, size and wavelength. The effect of the refractive index of the spherical particles on retro-reflection and the strength and angular distribution of reflected light can be precisely calculated from Mie's theory. For example, Table 1 shows calculations from Mie's theory for spherical particles with diameter of 0.5 μm to 5 μm and with a refractive index of 2.1. The irradiating wavelength is 500 nm (mid-visible) and the refractive index of the medium is 1.37 (human skin). Mie's theory was used to calculate the light scattering cross-section in μm² and a coefficient of back-reflection.

TABLE 1

| | Cross- | |
|---|---|---|
| Radius, | Section, | Back |
| (μm) | (μm²) | Reflection |
| 0.250000 | 0.854202 | 0.096035 |
| 0.272727 | 0.865080 | 0.066109 |
| 0.295455 | 1.005253 | 0.230000 |
| 0.318182 | 0.912985 | 0.164054 |
| 0.340909 | 0.994792 | 0.352563 |
| 0.363636 | 0.857336 | 0.387420 |
| 0.386364 | 0.947284 | 0.453141 |
| 0.409091 | 0.875968 | 0.604106 |
| 0.431818 | 1.081920 | 0.695207 |
| 0.454545 | 1.168800 | 0.737624 |
| 0.477273 | 1.559686 | 1.112604 |
| 0.500000 | 1.823330 | 1.229301 |
| 0.522727 | 2.341018 | 1.327851 |
| 0.545455 | 2.749733 | 2.751187 |
| 0.568182 | 3.159570 | 1.027433 |
| 0.590909 | 3.432383 | 3.643293 |
| 0.613636 | 3.641312 | 0.490531 |
| 0.636364 | 3.460876 | 2.007832 |
| 0.659091 | 3.651570 | 0.395240 |
| 0.681818 | 3.331581 | 1.114407 |
| 0.704545 | 3.466319 | 0.602399 |
| 0.727273 | 3.212945 | 1.156943 |
| 0.750000 | 3.455426 | 0.542248 |
| 0.772727 | 3.421422 | 1.697471 |
| 0.795455 | 3.918810 | 0.546222 |
| 0.818182 | 4.185365 | 2.012027 |
| 0.840909 | 5.375163 | 4.237389 |
| 0.863636 | 5.554469 | 2.722422 |
| 0.886364 | 6.120851 | 0.321047 |
| 0.909091 | 6.739395 | 0.003831 |
| 0.931818 | 7.151290 | 0.236304 |
| 0.954545 | 7.552325 | 0.563684 |
| 0.977273 | 7.654250 | 0.052019 |
| 1.000000 | 7.923596 | 1.202597 |
| 1.022727 | 7.615717 | 0.003093 |
| 1.045455 | 7.892309 | 1.888872 |
| 1.068182 | 7.418138 | 0.039752 |
| 1.090909 | 7.795960 | 2.296835 |
| 1.113636 | 8.537990 | 5.068417 |
| 1.136364 | 8.163077 | 1.813724 |
| 1.159091 | 8.537695 | 0.021052 |
| 1.181818 | 9.320707 | 1.536560 |
| 1.204545 | 10.108878 | 0.021054 |
| 1.227273 | 11.001527 | 2.341658 |
| 1.250000 | 11.798744 | 0.034436 |
| 1.272727 | 12.592628 | 3.894475 |
| 1.295455 | 12.983492 | 0.204513 |
| 1.318182 | 13.625775 | 4.784204 |
| 1.340909 | 13.417889 | 0.866004 |
| 1.363636 | 13.975428 | 5.445824 |
| 1.386364 | 13.830625 | 4.879027 |
| 1.409091 | 13.507415 | 2.692945 |
| 1.431818 | 13.177734 | 5.007472 |
| 1.454545 | 13.468979 | 1.354508 |
| 1.477273 | 13.481708 | 1.622545 |
| 1.500000 | 14.723757 | 2.726182 |
| 1.522727 | 15.033455 | 0.986294 |
| 1.545455 | 16.889629 | 4.024335 |
| 1.568182 | 17.196861 | 1.886613 |
| 1.590909 | 19.108079 | 4.214462 |
| 1.613636 | 19.151885 | 2.892182 |
| 1.636364 | 20.559224 | 4.526062 |
| 1.659091 | 20.247216 | 3.112136 |
| 1.681818 | 21.714072 | 7.576826 |
| 1.704545 | 21.699415 | 20.059965 |
| 1.727273 | 20.603793 | 12.213325 |
| 1.750000 | 20.001850 | 1.802121 |
| 1.772727 | 20.407207 | 9.647434 |
| 1.795455 | 20.210020 | 4.920243 |
| 1.818182 | 21.141477 | 6.084316 |
| 1.840909 | 21.668551 | 7.425834 |
| 1.863636 | 23.013854 | 4.652557 |
| 1.886364 | 24.517870 | 13.157777 |
| 1.909091 | 25.567726 | 5.558807 |
| 1.931818 | 27.619779 | 28.117990 |
| 1.954545 | 28.000047 | 6.946927 |
| 1.977273 | 28.575760 | 18.069465 |
| 2.000000 | 29.471907 | 3.872414 |
| 2.022727 | 28.884996 | 14.896184 |
| 2.045455 | 29.788250 | 4.322329 |
| 2.068182 | 28.526677 | 11.634326 |
| 2.090909 | 29.449068 | 6.304170 |
| 2.113636 | 28.284982 | 9.071041 |
| 2.136364 | 29.480639 | 6.280634 |
| 2.159091 | 29.203944 | 12.549098 |
| 2.181818 | 30.760251 | 3.123629 |
| 2.204545 | 32.162120 | 22.862156 |
| 2.227273 | 33.506243 | 2.009651 |
| 2.250000 | 35.241840 | 3.892219 |
| 2.272727 | 36.577758 | 5.717323 |
| 2.295455 | 37.503079 | 2.204915 |
| 2.318182 | 38.643466 | 2.343115 |
| 2.340909 | 39.358654 | 5.461561 |
| 2.363636 | 39.511260 | 1.795520 |
| 2.386364 | 40.071195 | 13.846648 |
| 2.409091 | 39.217666 | 0.673838 |
| 2.431818 | 40.094102 | 22.303574 |
| 2.454545 | 38.875149 | 1.198236 |
| 2.477273 | 39.577571 | 17.059726 |
| 2.500000 | 39.730453 | 3.351106 |

Table 2 shows calculations according to Mie's theory for particles with a refractive index of 1.5. All other parameters are the same as those used to calculate the results shown in Table 1.

TABLE 2

| | Cross- | |
|---|---|---|
| Radius, | Section, | Back |
| (μm) | (μm²) | Reflection |
| 0.250000 | 0.057110 | 0.000607 |
| 0.272727 | 0.081604 | 0.000180 |
| 0.295455 | 0.112892 | 0.000141 |
| 0.318182 | 0.152333 | 0.000734 |
| 0.340909 | 0.201018 | 0.000519 |
| 0.363636 | 0.259483 | 0.000008 |
| 0.386364 | 0.329489 | 0.000292 |
| 0.409091 | 0.412466 | 0.000585 |
| 0.431818 | 0.508662 | 0.000504 |
| 0.454545 | 0.619689 | 0.000149 |
| 0.477273 | 0.747051 | 0.000202 |
| 0.500000 | 0.891441 | 0.001626 |
| 0.522727 | 1.053642 | 0.001716 |
| 0.545455 | 1.234928 | 0.000236 |
| 0.568182 | 1.436742 | 0.002486 |
| 0.590909 | 1.659037 | 0.004784 |
| 0.613636 | 1.902605 | 0.001693 |
| 0.636364 | 2.168919 | 0.002184 |
| 0.659091 | 2.458184 | 0.006676 |
| 0.681818 | 2.770197 | 0.003773 |
| 0.704545 | 3.105600 | 0.000929 |
| 0.727273 | 3.465833 | 0.005055 |
| 0.750000 | 3.849188 | 0.003996 |
| 0.772727 | 4.255779 | 0.000054 |
| 0.795455 | 4.687212 | 0.001867 |
| 0.818182 | 5.141158 | 0.001633 |
| 0.840909 | 5.617977 | 0.000879 |
| 0.863636 | 6.116780 | 0.002791 |
| 0.886364 | 6.637160 | 0.000380 |
| 0.909091 | 7.178885 | 0.003490 |
| 0.931818 | 7.738020 | 0.010912 |
| 0.954545 | 8.317066 | 0.005097 |
| 0.977273 | 8.913523 | 0.006391 |
| 1.000000 | 9.523621 | 0.021157 |
| 1.022727 | 10.149276 | 0.014744 |
| 1.045455 | 10.786943 | 0.007346 |
| 1.068182 | 11.435497 | 0.023740 |
| 1.090909 | 12.092112 | 0.020783 |
| 1.113636 | 12.755851 | 0.005124 |
| 1.136364 | 13.425822 | 0.015569 |
| 1.159091 | 14.096643 | 0.016261 |
| 1.181818 | 14.769270 | 0.001307 |
| 1.204545 | 15.440571 | 0.006747 |
| 1.227273 | 16.108802 | 0.006861 |
| 1.250000 | 16.770355 | 0.000270 |
| 1.272727 | 17.423912 | 0.011769 |
| 1.295455 | 18.070610 | 0.008180 |
| 1.318182 | 18.701161 | 0.005591 |
| 1.340909 | 19.320146 | 0.032292 |
| 1.363636 | 19.923744 | 0.027737 |
| 1.386364 | 20.507448 | 0.015154 |
| 1.409091 | 21.074309 | 0.051061 |
| 1.431818 | 21.616178 | 0.051807 |
| 1.454545 | 22.139849 | 0.020888 |
| 1.477273 | 22.636627 | 0.048547 |
| 1.500000 | 23.106269 | 0.056612 |
| 1.522727 | 23.554698 | 0.016243 |
| 1.545455 | 23.970151 | 0.026967 |
| 1.568182 | 24.361089 | 0.036759 |
| 1.590909 | 24.720572 | 0.005126 |
| 1.613636 | 25.053106 | 0.012191 |
| 1.636364 | 25.356685 | 0.017501 |
| 1.659091 | 25.625806 | 0.000666 |
| 1.681818 | 25.874781 | 0.026682 |
| 1.704545 | 26.086784 | 0.029149 |
| 1.727273 | 26.276008 | 0.011582 |
| 1.750000 | 26.437105 | 0.060738 |
| 1.772727 | 26.569330 | 0.069205 |
| 1.795455 | 26.686193 | 0.029697 |
| 1.818182 | 26.765513 | 0.078474 |
| 1.840909 | 26.838476 | 0.098360 |
| 1.863636 | 26.885280 | 0.036221 |
| 1.886364 | 26.910744 | 0.058250 |
| 1.909091 | 26.934903 | 0.083133 |
| 1.931818 | 26.929681 | 0.023045 |
| 1.954545 | 26.932570 | 0.021785 |
| 1.977273 | 26.915222 | 0.039833 |
| 2.000000 | 26.898570 | 0.004217 |
| 2.022727 | 26.890992 | 0.013577 |
| 2.045455 | 26.865076 | 0.020556 |
| 2.068182 | 26.869992 | 0.001962 |
| 2.090909 | 26.864058 | 0.047289 |
| 2.113636 | 26.882866 | 0.052130 |
| 2.136364 | 26.917153 | 0.018541 |
| 2.159091 | 26.958307 | 0.084445 |
| 2.181818 | 27.050493 | 0.098647 |
| 2.204545 | 27.134160 | 0.032190 |
| 2.227273 | 27.278340 | 0.077335 |
| 2.250000 | 27.442490 | 0.101041 |
| 2.272727 | 27.636439 | 0.024565 |
| 2.295455 | 27.897053 | 0.032018 |
| 2.318182 | 28.159666 | 0.052356 |
| 2.340909 | 28.514964 | 0.006127 |
| 2.363636 | 28.882424 | 0.010380 |
| 2.386364 | 29.312329 | 0.011391 |
| 2.409091 | 29.817064 | 0.006153 |
| 2.431818 | 30.331643 | 0.053524 |
| 2.454545 | 30.960889 | 0.033237 |
| 2.477273 | 31.596919 | 0.028422 |
| 2.500000 | 32.329533 | 0.123769 |

The results in Tables 1 and 2 show that retro-reflective particles comprising a material with a refractive index of 1.5 provide about 1/1000 of brightness of particles comprising a material with a retro-reflective index of 2.1. Thus, for the retro-reflective tissue marking to be bright, the particles should have a high refractive index.

Further, for high-index particles, such as the glass spheres with a refractive index of 2.1, colors will result from even small changes in particle size and viewing angle. Thus, iridescent tissue markings can be made in different shades without using different materials.

Since appearance of a retro-reflective tissue marking strongly depends on particle size and shape, the retro-reflectivity, and consequently, the tissue marking's color can be removed. When the particles are pulverized by, for example, an infrared pulse, the colors will no longer be visible.

For use in tissue, particles with a refractive index greater than about 1.6 are useful and particles with a refractive index in the range from about 1.6 to about 2.4 are preferred. Titanium dioxide and various high-index glasses are such materials. Titanium dioxide is commonly used in paints and sunscreens in amorphous or microcrystalline form, and can be made as pure microspheres, or coated onto a glass microsphere or nanosphere substrate. Very high-index glass microspheres are commercially available as bright retro-reflector additives to paints, clothing materials, etc. Solution-based chemical precipitation can be used to make titanium dioxide, glass, silica, or polymeric nanoparticles by different manufacturing processes. Commercially-available nanoparticles, some of which act as retro-reflectors are summarized, for example, at the website http://solgel.com/precursors/nano.

The variable appearance retro-reflective tissue markings of the present invention are extremely versatile. These markings can be invisible under normal lighting conditions and/or colored to affect the appearance of the marking upon exposure to incident electromagnetic radiation. Also, these markings can retro-reflect different types of electromagnetic radiation, such as, for example, ultraviolet, visible and/or infrared.

Materials for Retro-Reflective Particles

Commercially available transparent spheres are made of various glasses, plastics or other organic polymers, with refractive indexes ranging from about 1.4 to about 2.1. As the refractive index of the sphere increases, so does its retro-reflectance. Excellent retro-reflection when imbedded in tissue may occur in a sphere with a refractive index greater than about 2. Noticeable retro-reflection, for example, in human skin will occur from spheres with a refractive index in the range from about 1.6 to about 2.4, as mentioned above.

Plastic or other organic polymer spheres have lower refractive index than most glasses. If the refractive index of the sphere is 1.5, which is about the refractive index of pure silica (SiO₂), not much retro-reflection is expected to occur when the sphere is placed in the dermis. Such silica spheres are commercially available in the small size range generally preferred for tissue markings.

However, one cannot simply buy an off-the-shelf product and inject it into the skin with the expectation of making an excellent tissue marking. While commercially available high-index glasses, which are, for example, used to make embedded-retro-reflective spheres for paint additives (e.g., Flex-o-Lite ultra-high index glass beads; n=2.1) are available in sizes as small as 40 μm, they are considerably larger than the ideal size for use as a tissue marking particle. Highway signs also use glass sphere retro-reflectors. However, these glass spheres are very large compared with most tattoo particles, typically from about 0.05-1 mm. Moreover, the commercially available spheres are not necessarily sterile, clean, or appropriate in other ways for injection into living tissue.

Spheres of both the desired size and refractive index ranges can be manufactured as needed. The microparticles can be manufactured of any material with a sufficiently high index that is capable of being made into a microparticle of appropriate dimensions. In general, as discussed above, the microparticles should have a refractive index of n≈1.6-2.4. Examples of high refractive index materials include high-index glasses, e.g., BK7 (n≈1.5); LaSFN9 (n≈1.9); SF11 (n≈1.8); F2 (n≈1.6); BaK1 (n≈1.6); barium titanate (n≈1.9); blue high-index glass (n≈1.6-1.7); TiO₂—BaO (n≈1.9-2.2); borosilicate (n≈1.6); or chalcogenide glass (n≈2 or higher). The microparticles can consist of cubic high-index crystals or other inert, insoluble, transparent minerals, e.g., sapphire (n≈1.8); diamond (n≈2.4); cubic zirconium (n≈2.2); zirconium silicate (n≈1.8-2.0); ruby (n≈1.8); and/or high-index transparent polymers such as crosslinked polystyrene (n≈1.6). Nanocomposites can also be used, e.g., iron sulfides and poly(ethylene oxide) (PEO) (n≈2.5-2.8).

Methods for Making Particles

As disclosed above, certain variable appearance tissue materials may be encapsulated to provide a tissue marking of the present invention. Using known encapsulation methods, including those described herein, it is possible to encapsulate one or more variable appearance materials within one or more biologically inert, substantially transparent coating materials described above to create a palette of inert, indispersible variable appearance particles for implantation into tissue to create permanent markings that can be removed on demand.

The optimal method for producing a desired particle generally depends on the properties of the specific materials used, e.g., core material and the coating; this method, in turn, determines the morphology, size, and surface characteristics of the product. In some particles of the invention, the core material can be a solid particle, a concentrated liquid solution, or even a gas (in any case it can include other inert materials such as buffers, diluents, carriers, and binders), but is in most cases hydrophilic. The coating material is applied in free-flowing form, for example, liquid (solvated, monomeric, or melted) or gas/plasma, and is hardened through several processes (such as evaporation, polymerization, cooling) to form a solid shell.

Four classes of microencapsulation methods, characterized by similar technique and apparatus, are useful in the current invention. In the first class, referred to herein as “aerosol collision,” aerosolized droplets or core particles (which include variable appearance material(s)) and coating material are made to collide, and then the coating is hardened. In the second class, referred to herein as “emulsion spraying,” an emulsion of core material in coating material is atomized (into a vacuum, gas, or liquid), and then the coating is hardened. In the third class, referred to herein as “chamber deposition,” coating material in a gas or plasma (very hot ionized gas) phase is deposited onto a solid core particle to form a solid shell. In the fourth class, referred to herein as “in situ encapsulation,” a mixture containing the core material and coating material in the same or different emulsion phases (depending on the technique) is prepared so that coatings are formed by polymerization or seeding out around core droplets, and then the microcapsules are separated. All four classes are capable of producing particles within the 50 nm to 100 micron size range which are indispersible.

The coating materials and variable appearance materials described herein can be prepared by using known methods as an example, but the requisite specific particle sizes made of appropriate materials are not readily available. To prepare solid variable appearance core particles of a desired size, bulk dry material can be ground and/or mesh-sifted or vacuum filtered (or prepared by any other suitable conventional means) as described in Standards 5 and 32 of the Metal Powder Industries Federation's Standard Test Methods for Metal Powders and Powder Metallurgy Products, 1993-1994 edition. Variable appearance nanoparticles can also be prepared from appropriate materials in this manner. Materials serving as buffers, diluents, carriers, binders, etc., may be added at this stage to change the solubility, perceived color, viscosity, mobility characteristics, etc., of the variable appearance preparation.

Absorption components 40 of a desired solid material and size can be prepared as described above for the variable appearance material, and can be mixed with melted coating materials or into liquid variable appearance preparations (which can be dried, reground and sifted to provide solid variable appearance core particles incorporating additional absorption components 40).

Sub-particles 70 and 90 can be prepared in the same manner as particles and then encapsulated along with other desired core elements, usually using a method that produces significantly larger final particles.

If desired, a secondary or higher order coating can be added to particles, for example, to reduce shell permeability or to improve particle suspension in liquid carriers for tissue marking inks. This can be accomplished by methods in any of the microencapsulation classes defined above, particularly chamber deposition and in situ encapsulation.

A useful microencapsulation method in the aerosol collision class is described in U.S. Pat. Nos. 3,159,874; 3,208,951; and 3,294,204 to Langer and Yamate. In this method, opposite voltages are applied to two reservoirs containing, respectively, the heated hardenable liquid coating material and a liquid core material. The materials are atomized or aerosolized into a common chamber using high-pressure air streams that produce submicron particles of about equal size. The opposite charges of the particles cause attraction and collision, resulting in the formation of neutral coated particles which can then be cooled below the coating hardening temperature.

Appropriate materials for use in this method must be able to hold a charge, they must wet each other, and the surface tension of the core material must be higher than that of the coating material. Suitable coating materials include natural and synthetic waxes, and specifically hard waxes like carnauba wax. Core materials can be hydrophilic liquids or solids that will hold a charge (such as glycerin into which a wavelength frequency up-converting material may be mixed). The resulting mostly unagglomerated 0.5 to 1.0 micron particles are nonporous and are stable for long-term use as tissue markings.

A useful microencapsulation method in the emulsion spraying class is disclosed in U.S. Pat. No. 4,675,140 to Sparks et al. Solid or viscous liquid particles of core material in a prepared size range, for example, about 20 microns, can be mixed with a liquid (melted or solvated) encapsulation coating material and dispensed onto a rotating disk spinning at a speed that allows coated particles of a total size within a tight range to be flung off the apparatus into a collection chamber (for correlation between disk rotation speed and final particle size, see the formula in column 11, line 64, of the Sparks et al. patent). Materials appropriate for coating lyophilized or viscous liquid hydrophilic variable appearance cores include synthetic and natural waxes (such as carnauba wax) and organic polymers D solvated in organic solvents. Coated particle sizes as small as about 25 microns can be achieved by this process.

Another example of a useful method in the emulsion spraying class is described in U.S. Pat. No. 5,589,194 to Tsuei. In this method, approximately one part hydrophilic, solid particles to be encapsulated are suspended in about two parts meltable coating material (such as carnauba wax) at a temperature above the coating materials melting point to form an emulsion. This step can be performed in a heated agitator (such as a turbine stirrer) and the suspension is stirred until an emulsion is formed. This emulsion is then loaded into a pressurized reactor, and a stream of the emulsion is forced into a temperature-controlled quenching agent (such as water) allowing formation and hardening of individual droplets into coated microspheres.

Other emulsion spraying methods for forming variable appearance material-containing particles include the use of rotating centrifugal force spray heads to direct emulsions of variable appearance materials dispersed in solvated organic polymers into a cooled liquid, gas or vacuum (such as in U.S. Pat. No. 3,015,128 to Somerville).

In most emulsion spraying processes, a significant percentage of agglomerated products can be formed. When solvents are evaporated to form hardened coatings, the resulting particles tend to be less regular and smooth, appearing wrinkled and/or collapsed compared to particles produced by other processes or in these processes using hardenable waxy matrices. Nevertheless, these unevenly shaped particles, which may contain multiple variable appearance cores or pockets as shown in FIG. 5, are suitable for use in tissue markings.

A general description of a microencapsulation method in the chamber deposition class is disclosed in U.S. Pat. No. 5,718,753 to Suzuki. A substantially uniform coating of a material can be deposited onto minute solid particles of 0.05 microns and larger at thicknesses in the range of 0.01 to 0.1 microns and greater using standard vacuum deposition or sputtering techniques. Several modifications to standard vacuum deposition apparatus can be made to achieve this end, including providing for agitation of the particles in the chamber to receive a more even coating on all sides (such as by using acoustic frequency vibrations). Metal oxide materials (such as silica) are routinely deposited using such apparatus. The coating material is brought to its sublimation point by varying temperature and pressure, and the resulting gas is deposited, coating solid core particles in the chamber. To improve coating efficiency and uniformity, the dry core particles and gas can be ionized so the coating, for example, silica, is attracted to the solid.

Similar chamber deposition methods have been developed for coating solid particles with inert polymeric films (such as in U.S. Pat. Nos. 5,288,504 and 5,393,533 to Versic). A vacuum deposition apparatus is used to deposit a parylene (such as para-p-xylylene) or a fluorocarbon (such as TEFLON®) by pyrolysis of a monomeric, usually gas-phase material (such as di-para-xylylene in the case of xylylene deposition, or hexafluoropropylene oxide in the case of TEFLON® deposition). Polymerization of these coating materials onto the surface of relatively cool small core particles occurs spontaneously. As in vacuum deposition or sputtering of metal oxides onto small particles, the core particles can be agitated to ensure the polymer is deposited evenly over their entire surface. The procedure can be repeated until the desired coating thickness is obtained. Coating thicknesses of under 1 micron of xylylene have been reported to give controlled release of the core substance; thicker walls offer greater protection to a variable appearance core. Once polymerized, both xylylene and TEFLON® are extremely inert materials approved for use in the body by the FDA.

Similar results can be achieved using sputtering apparatus to apply metal oxide coatings as described in U.S. Pat. No. 5,506,053 to Hubbard. In this method, a sputtering cathode is used to sputter a coating onto solid core particles of about 5 microns and larger. One feature of particles coated using chamber deposition methods is that the majority of the coatings include significant pores. The presence, number, and size of pores can be controlled by the coating thickness and by varying the conditions for coating deposition. In certain embodiments, porous particles are advantageous.

Useful microencapsulation methods in the in situ encapsulation class are well known in the art (see, for example, U.S. Pat. No. 5,234,711 to Kamen). The advantage of in situ encapsulation methods is that they use only standard glassware and laboratory apparatus. The coating material polymers that are useful in these in situ methods must be used with care to avoid unpolymerized species or residual reactive polymerization initiators in the resulting particles, either of which may have undesirable toxicological profiles. Although many organic polymer encapsulation materials have the potential to raise allergic reactions when implanted, biocompatible organic polymers approved for use in medical devices by the FDA (such as Epo-Tek™ 301 and 301-2 manufactured by Epoxy Technology) are acceptable materials that can be applied using these standard methods.

Processes that use extremely toxic organic solvents are disfavored because particles bearing traces of organic residues can induce local toxic reactions when implanted into tissue. This risk can be reduced by optimizing the manufacturing process and by purifying the resulting particles, for example, by filtration and/or washing.

In general, aqueous or hydrophilic core materials are suspended in a hydrophobic and/or organic coating solution to prevent solvation of the core phase into the coating phase. The dispersed core phase can contain materials (such as a catalyst) that induce polymerization of the coating material. For example, when an aqueous solution with variable appearance materials is dispersed in an organic phase containing cyanoacrylate monomers (which polymerize in the presence of water or base), water acts as a catalyst and cyanoacrylate coatings form around the liquid cores.

Another example in the in situ encapsulation class is disclosed in U.S. Pat. No. 5,690,857 to Osterried, wherein solid substances that are insoluble in sodium water glass solution can be coated with an outer layer of an inorganic metal salt. Using this bench-top procedure, a (hydrophilic) variable appearance material previously encapsulated in an organic polymer, or a hydrophobic liquid or solid variable appearance material, can be coated with SiO₂ by suspending the particles in water, adding sodium water glass solution, and manipulating temperature, pH, and other seeding conditions to result in the formation of uniform coatings around the cores. Particles coated in this manner can exhibit improved suspension in aqueous vehicles for use as tissue markings.

Other in situ encapsulation methods can be used as long as they are capable of encapsulating the particular variable appearance core, which is in many cases hydrophilic. These other methods, many of which are described in The Kirk-Othmer Encyclopedia of Chemical Technology, 3rd ed., Vol. 16, pages 628-651, include in situ polymerization (as commonly defined and in contrast with the broader term, in situ encapsulation, used herein), simple and complex coacervation, polymer-polymer incompatibility, interfacial polymerization, and others.

When porous coatings are desired (such as to control the release of encapsulated variable appearance materials), slight modifications can be made in the standard techniques outlined herein and/or specific materials can be used. Porous particles can be prepared by in situ encapsulation using certain coating materials (such as melamine formaldehyde), which have an open “netlike” structure once polymerized. In aerosol collision or emulsion spraying processes a volatile component can be added to melted coating material before capsule formation and later evaporated to leave pores, for example, in wax coatings (such as described in U.S. Pat. No. 5,589,194 to Tsuei and No. 5,204,029 to Morgan). A porous coating can be made by chamber deposition methods as described above, for example, by applying very thin coatings of a metal oxide (such as described in U.S. Pat. No. 5,376,347 to Ipponmatsu).

Non-encapsulated particles composed of frequency up-converting material, such as glass microbeads or crystals, may be prepared by pulverizing glass or crystalline bulk by, for example, grinding, ultrasound, shock wave treatment, or particle bombardment. Then, the resulting particles are separated by size.

Ultra-high index glass or other transparent high-index materials can be used to make spherical particles in the appropriate size range depending on the type of tissue designated for marking. These particles can be made, for example, by centrifugal dispersion of molten material from a very rapidly spinning disc, as known to those skilled in the art. The particles can be sorted for size and quality, for example, by centrifugation or filtration.

These and other known methods can be used to create the particles of the present invention.

Particles intended for implantation in the body are preferably sterile. To ensure sterility, the manufacturing process can be carried out under sterile conditions, the finished particles can be exposed to gamma rays, or, if these conditions will not damage the particles, they can be exposed to chemicals, heat and/or pressure (such as autoclaving) sufficient to achieve sterilization.

Methods of Use

Particles of the invention, regardless of how prepared, can be used to create tissue markings for cosmetic, identification, and other purposes. Particles are suspended in a liquid carrier, for example, alcohol, water, and/or glycerin, to form a tissue marking ink in the same manner as standard tattoo pigments.

The inks can be implanted into skin or similar superficial tissue with an electromagnetic coil tattooing machine (such as that disclosed in U.S. Pat. No. 4,159,659 to Nightingale); a rotary permanent cosmetics application machine (such as that disclosed in U.S. Pat. No. 5,472,449 to Chou); or with any manual tattooing device (such as the sterile single-use device marketed by Softap Inc., San Leandro, Calif.).

Alternatively, the inks can be implanted using a non-invasive method, for example, as described in U.S. Pat. No. 5,445,611 to Eppstein. This non-invasive technique is well-suited to create an even tone of pigment over a relatively large body surface area. For example, using this method a removable sun tan can be made with particles of the invention.

Tissue markings in skin must be properly placed to provide permanent markings. Skin is composed of the outermost epidermis, generated by the constantly dividing stratum basale, and the underlying dermis. Dermal cells, such as fibroblasts, mast cells, and others, which do not generally replicate, are located within a resilient proteinaceous matrix. It is the upper dermis, below the stratum basale, into which the particles are implanted to provide a tissue marking (such as a tattoo).

After implantation by any of the foregoing techniques, particles in the dermis form part of a permanent tissue marking if they are phagocytosed by dermal cells (most likely for particles under about 5 microns), or if they remain in the extracellular matrix (most likely for particles 5 microns and larger). Some particles will inevitably be engulfed by immune cells and relocated from the area during the healing process.

In addition to skin, particles of the invention can be implanted into a wide variety of living tissues comprising relatively stationary, infrequently-replicating cells. For example, the particles can be implanted into the internal surfaces of the body that are contiguous with the external skin, including, but not limited to, the inner surfaces of the mouth and lips, the gums and tongue, inner surfaces of the eyelid, muscles, tendons and the tissues lining internal body passages (such as the nasal, ear, anal, urethral, and vaginal passages, and the gastrointestinal tract). Other tissues that can be marked include the tissues of and/or under the fingernails and toenails, the dentin of the teeth, and the colored iris and white sclera of the eye.

As a result of their versatility, the particles can be used to produce a wide variety of cosmetic tissue markings including decorative artistic tattoos that are removable and revisable, as well as cosmetic makeup (also known as cosmetic tattooing, permanent makeup, micropigment implantation, and variations on these names) that is permanent as long as the wearer desires it.

In addition to marking skin, the particles can be used to produce new cosmetic markings in other tissues. It is especially important that these new types of markings are removable to allow risk-free experimentation. For example, the particles can be added to the tissue of and/or under the fingernails and/or toenails, for example, to create solid colors, patterns, or designs for decorative purposes when exposed to specific wavelengths.

Identification markings made with the particles can be changed, updated, and/or removed. In some cases, selectively detectable (such as normally invisible) particles may be advantageous. Some examples of markings to fill identification needs include markings to assist tracking bodily sites of medical interest in external and superficial internal tissue, for example, marking a radiation therapy field on the skin, or marking a colon polyp in the intestine which can subsequently be monitored endoscopically; identification markings for humans, for example, emergency information regarding an individual's medical history, “dog-tags” on military personnel, and identification markings on newborn babies to ensure no hospital mix-ups; and identification markings for animals (such as wild animals, livestock, sport/show animals, and pets), for example, information markings for the return of lost pets.

Removal Methods

Certain types of tissue markings of the invention can be removed by applying specific energy (such as electromagnetic radiation), preferably using energy by which the particles used to create the tissue marking were designed in advance to be removed. Certain particles are designed to be ruptured, releasing into the bodily fluids the dispersible variable appearance material, which are then metabolized or relocated from the tissue marking site. Other particles are designed to remain intact while the variable appearance properties of the encapsulated material within the particle are altered, rendering the tissue marking undetectable.

For removal of tissue markings created using particles of the invention described in detail herein, the marking site is exposed to a specific type of energy (such as electromagnetic radiation by which the particles comprising the marking were designed in advance to be removed). The energy is applied using an external source (such as a laser or flash-lamp at specific wavelengths) at a specific intensity and for a controlled length of time. The exposure can be administered in one or several pulses. A range of electromagnetic radiation, for example, ultraviolet, visible, infrared, microwave, and radio wave, may be suitable for removing the tissue markings. The preferred wavelengths are those that the particles were specifically designed in advance to absorb, for example, by use of specific radiation absorbing materials within the particle.

The particles are designed in advance to be removed using devices emitting safe forms of energy, which are minimally absorbed by ubiquitous energy absorbing substances normally present in the body. These substances include water, which absorbs at 1800 nm and greater; melanin, which absorbs at up to about 1100 nm, but absorbs far less energy at wavelengths over 800 nm; and oxyhemoglobin, which has a maximal absorbency in the range of 400 to 600 nm. Thus, particularly for condition-dependent appearance markings, a desirable spectral region is the near-infrared, specifically about 800 to 1800 nm. As noted earlier, many useful materials are available that absorb in this near-infrared range. Certain types of microwaves and radio waves can also be very specific and safe.

In theory, external devices producing safe energy other than electromagnetic radiation can be used to remove tissue markings that are specifically designed in advance for removal by this energy. For example, intense ultrasound waves are capable of causing cavitation, or the rapid expansion and collapse of gas bubbles, within the tissue. The threshold for initiating cavitation depends on the local intensity and frequency of ultrasound waves, and on the material's acoustic, mechanical, and thermal properties. Cavitation is initiated more easily when ultrasound waves interact with an existing gas bubble, causing the absorption and scattering of waves. Stable gas microbubbles have recently been employed, for example, as contrast agents for medical ultrasound imaging. It is theoretically possible to construct variable appearance particles that contain stable encapsulated gas bubbles designed in advance to enhance ultrasound-induced cavitation and rupture of the particles. Electromagnetic radiation, however, can supply more energy specifically to the particles. It is the preferred energy for removal and is therefore described in greater detail herein.

Particles of the invention can be designed in advance such that multiple variable appearance particles all selectively absorb radiation of the same wavelength regardless of their apparent color. This feature is accomplished by using common radiation-absorbing material(s) in combination with other particle materials (such as coating 20 or specific absorption component(s) 40), which enables removal of diverse particles using a common energy type(s). For example, a tissue marking of the invention can be designed such that all variable appearance particles are neutralized or removed in a treatment with a Nd:YAG laser emitting 1064 nm pulses, which target a common iron oxide, metal or carbon absorption component 40 in all tissue marking particles.

Dispersible variable appearance materials in particles such as those constructed according to FIGS. 1, 2, 3, and 5 can be designed in advance to be removed using electromagnetic radiation, which ruptures the particles, thereby releasing the variable appearance materials. Radiation absorption by an absorbing coating 20, variable appearance material(s) 30 or 32, or an additional absorption component(s) 40 located in the coating or the variable appearance core (depending on the particle's design) causes the coating to rupture. Cells in the tissue may or may not be ruptured concomitantly, depending on the amount of energy applied and the pulse length in which it is delivered. After irradiation, variable appearance material dispersal occurs through physiological processes in both cases and the marking is removed from the tissue. The total systemic dose of the released variable appearance materials is generally low following a removal treatment.

The amount of energy per unit area (fluence) required to rupture particles 10, 50, 60, and 100 made with given target materials (such as a specific absorption component 40 or an absorbing coating 20) can be determined. The fluence (E) can be estimated based on the optical absorption coefficient (μa) and the heat capacity (ρc), which are known for different materials, and the required change in temperature to cause breakage (ΔT), from the following equation:
E=ΔTρc/μa.

The temperature change (ΔT) can be set, for example, at about 280° C. to provide thermal excitation to over 300° C., at which water very violently expands, for vaporization/rapid expansion of an aqueous core, ensuring destruction of particles; other values can be chosen for other types of core materials. Mechanical stress induced by rapid heating and/or differential expansion of the coating can provide additional mechanisms for particle rupture. Heating of the coating, the variable appearance materials, absorption component 40, or any combination can be responsible for particle rupture and therefore the value of ΔT required for rupture of differently constructed particles may vary considerably. For example, for particles made with a coating of Schott filter glass KG-3, a fluence of about 20 Joules/cm² is sufficient to achieve a 100° C. temperature change based upon the above equation using the known optical absorption coefficient for KG-3 of about 20 cm⁻¹ at a wavelength of 1064 nm (such as from a Nd:YAG laser) and its heat capacity of about 4 Joules/cm³/° C.

In general, visible and near-infrared fluences of about 0.1 to 30 Joules/cm² are applied, and are well tolerated by the skin. 1.0 to 5.0 Joules/cm² are most suitable, but higher laser fluences that do not injure the tissue can be used, and lower fluences can be used as long as they rupture the particles.

The preferred electromagnetic radiation pulse duration used to effect mechanical rupture or thermal alteration of a particle is approximately less than or equal to the thermal relaxation time of (τᵣ) of the particle (see Anderson, R. R. and J. A. Parrish (1983). Science 220: 524-527). To a close approximation, τᵣ in seconds is equal to d², where “d” is the target diameter in millimeters. This pulse duration results in thermal confinement at the particle, reducing secondary damage to surrounding tissue. For example, a 100 nm (104 mm) diameter particle (such as a 100 nm absorption component 40 in a 10 micron diameter particle) is preferably treated with a pulse duration of less than or equal to about (10⁻⁴)² or 10⁻⁸ seconds (10 nanoseconds). In general, the energy can be delivered in pulses ranging from 0.1 to about 100, 500, or 1000 nanoseconds. Typical Q-switched lasers operate in this range. Within this range, pulses of 0.5 to 100 nanoseconds are preferred.

Current radiation systems useful to remove tissue markings according to the first removal method include Q-switched near-infrared lasers presently used in standard tattoo removal treatments (such as the 1064 nm Q-switched Nd:YAG or 760 nm Alexandrite lasers).

In removing tissue markings made using particles that are rendered invisible without being ruptured, as described hereafter, the tissue can experience even less trauma than in the embodiments described above. Cells are unlikely to be damaged during tissue marking removal, and many cells may not be affected in any substantial way or even to be “aware” of treatment.

Neutralizable variable appearance particles (such as those constructed according to FIG. 4) are designed in advance to be removed using electromagnetic radiation, which ruptures sub-particle(s) 90 containing neutralizing agent(s) 100 without rupturing the entire particle 80. This method can be practiced by following the guidelines above to determine how much of a given type of energy should be applied to rupture the sub-particle through absorption by coating 95, specific absorption component 40, and/or neutralizing agent 100. The electromagnetic radiation should be administered using a device emitting wavelengths that are not strongly absorbed by the coating 20 or variable appearance material(s) 34. A conservative dose should always be administered in this case to avoid rupturing the entire particle which could cause leakage of reactive compounds into the tissue. Once the neutralizing agent mixes with the variable appearance material, the tissue marking will become undetectable at completion of (1) the oxidation reaction, or (2) the pH transition.

Photobleachable variable appearance particles (such as those constructed according to FIGS. 1, 2, 3, or 5) are designed in advance to be removed using electromagnetic radiation that affects the specific variable appearance material(s) 30 or 32 without rupturing the entire particle 10, 50, 60, or 110. Once exposed to that radiation in an appropriate dosage, the variable appearance material loses its ability to be detectible and/or visible because of an irreversible chemical transition or decomposition, and the tissue marking is removed. Again, the radiation administered should not be strongly absorbed by coating 20. Further, it is desirable that common electromagnetic radiation sources, such as sunlight, not trigger the irreversible chemical transition or decomposition, which lead to removal of variable appearance tissue markings. Two-photon absorption of short, high-intensity pulses from lasers or IPLs can be used to trigger photobleaching of otherwise photostable materials, including benzophenones, ketones, and radical generators, which absorb below about 300 nm.

Thermolabile variable appearance particles (such as those constructed according to FIGS. 1, 2, 3, or 5) are designed in advance to be removed using electromagnetic radiation tuned to heat the variable appearance core(s) without rupturing the entire particle 10, 50, 60, or 10. For example, radiation may be absorbed by and heat the variable appearance material to or above a specific temperature at which its variable appearance properties are changed or lost either by an irreversible chemical transition or tertiary structure disruption.

Some patients may desire partial removal of a tissue marking, which is also achieved by irradiation. Incomplete removal can be achieved, for example, by administering lower doses of radiation to affect only a fraction of particles, or by only treating certain areas of the tissue marking. It may be desirable, for example, to reduce the size of the marking; to remove a portion of a marking including a smaller mark, symbol, text, or identifying information; to reduce the intensity of a marking; or to transform the appearance of the tissue marking.

In the event that a new tissue marking is desired to replace an existing marking, radiation is used to remove all or part of the original marking. New particles are then implanted into the tissue. The process could be used to update marks (such as bar codes), symbols, text, or identifying information, for example, to change a phone number marking on a pet after a move; to rework or refresh the appearance of the remaining tissue marking, for example, to add details to an artistic tattoo after regions have been removed to reduce the tattoo size; or to replace completely the original marking with a new tissue marking.

A retro-reflective tissue marking comprising, for example, IR-absorbing glass particles, can be removed by using an IR pulse to shatter the particles.

### EXAMPLES

The following example illustrates, but does not limit the scope of, the invention, which is defined by the claims.

### Example 1

Ultra-high index glass spherical particles in the 0.5-5 micrometer size range are prepared by centrifugal dispersion of molten material from a very rapidly spinning disc. The particles are sorted for size and quality by centrifugation, sterilized, suspended at high concentration in a water/glycerin medium, and injected into the superficial dermis with a standard hypodermic needle. For the purposes of verifying biocompatibility, longevity, appearance and retro-reflection properties, the tissue markings can be implanted in animals such as rats, mice, pigs, etc. prior to humans.

Healing of the skin is allowed for about one month. Skin reactions to the tattoo are observed for signs of persistent inflammation, elimination and stability of the tattoos. Microscopic examination of stained skin biopsies is performed to verify the location and minimal skin reaction to the tattoo material.

The quality of retro-reflection of the tattoos is observed and measured using a collimated light source and a digital camera apparatus to monitor the appearance and angular distribution of reflected light. The amount of material needed for providing a given intensity of retro-reflection is measured by a variation of the concentration and/or amount of material injected. Retro-reflection intensity and angular distribution among spheres of different sizes and refractive indexes are compared.

A schematic illustration of a set-up that can be used to measure retro-reflective strength of a tissue marking is shown in FIG. 8. A beam of light is projected from a light source 1 using a beam splitter 2 onto particles 3, which are implanted in tissue 4. The light source 1 can be a laser or any other stable, collimated light source that emits light at a specific wavelength or a range of wavelengths.

The beam is reflected by the particles 3 toward a CCD (charge-coupled device) camera. This camera is movable, as shown by the dotted line, to detect light from the particles 3 at different angles of reflection 0. As shown in FIG. 8, the reflection is measured at θ=0°. In this configuration, a beam splitter 2 that allows the CCD camera to view retro-reflection is used.

The CCD camera captures the retro-reflected light and sends information to a digitized image capture, which is connected to a computer. The computer can process the information from the digital image capture and quantify the distribution of reflection intensities at different angles θ in terms of, for example, pixel intensity (e.g., 0-256 for an 8-bit image digitizer).

This distribution of reflection intensities at different angles of reflection can be used to evaluate retro-reflective strength of a particular tissue marking. The intensities can be compared using, for example, a polar plot.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A particle for use in a variable appearance tissue marking, wherein the particle comprises a variable appearance material that varies in any one or more of appearance via frequency up-conversion, retro-reflection, metachromasia or a change in an oxidation state.

2. The particle of claim 1, wherein the variable appearance material varies in appearance via a frequency up-conversion.

3. The particle of claim 2, wherein the variable appearance material comprises sodium yttrium fluoride.

4. The particle of claim 2, wherein the variable appearance material comprises erbium yttrium fluoride.

5. The particle of claim 1, wherein the variable appearance material varies in appearance via a change in the oxidation state of the variable appearance material.

6. The particle of claim 5, wherein the variable appearance material comprises a dye that is reversibly oxidized or reduced.

7. The particle of claim 6, wherein the dye comprises methylene blue.

8. The particle of claim 5, wherein the variable appearance material comprises a transition metal oxide, a transition metal hydroxide, or an aqueous solution of a transition metal salt or complex.

9. The particle of claim 8, wherein the transition metal is Cu, Fe, Co, Mn, or Cr.

10. The particle of claim 1, wherein the variable appearance material varies in appearance via metachromasia.

11. The particle of claim 10, wherein the variable appearance material comprises a phenothiazinium dye or a cyanine dye.

12. The particle of claim 10, wherein the variable appearance material comprises an ionic dye that binds in solution with a polyectrolyte.

13. The particle of claim 12, wherein the particle further comprises a polyelectrolyte that binds in solution with the variable appearance material.

14. The particle of claim 12, wherein the polyelectrolyte comprises a protein, a nucleic acid, or a polysaccharide that comprises an ionized group.

15. The particle of claim 12, wherein the ionic dye comprises toluidine blue, brilliant cresol blue, or methyl green pyronine.

16. The particle of claim 10, wherein the variable appearance material comprises a fluorescent nucleic acid stain that exhibits enhanced fluorescence or an emission spectra shift when bound to a nucleotide.

17. The particle of claim 16, wherein the fluorescent nucleic acid stain comprises ethidium bromide or acridine orange.

18. The particle of claim 1, wherein the particle is from about 50 nm to about 100 μm in size.

19. The particle of claim 1, wherein the particle is indispersible in tissue, is substantially biologically inert, or both.

20. The particle of claim 1, wherein the variable appearance material is indispersible in tissue and/or is substantially biologically inert.

21. The particle of claim 1, wherein the variable appearance material is insoluble and has a size and configuration such that it is physically relocated from the marking by biological processes when released into tissue.

22. The particle of claim 1, wherein the particle is sterilized.

23. The particle of claim 1, wherein the variable appearance material comprises a substance approved by the United States Food and Drug Administration for use in humans.

24. The particle of claim 1, wherein the particle is visible when exposed to infrared radiation, near-infrared radiation, or both.

25. The particle of claim 24, wherein a wavelength of the radiation is from 700 nm to 1300 nm.

26. The particle of claim 24, wherein the particle is visible only when exposed to near-infrared radiation.

27. The particle of claim 26, wherein a wavelength of said near-infrared radiation is about 980 nm.

28. The particle of claim 27, wherein the particle emits green light when exposed to said wavelength.

29. The particle of claim 1, wherein the particle comprises a coating and a core enveloped within the coating, wherein the core comprises the variable appearance material, and wherein the core is detectable through the coating.

30. The particle of claim 29, wherein the variable appearance material is dispersible and is dissolved when released into tissue.

31. The particle of claim 29, wherein the variable appearance material is dispersible and is metabolized when released into the tissue.

32. The particle of claim 29, further comprising an absorption component that absorbs a specific energy and that is located in the coating, in the core, or in both.

33. The particle of claim 32, wherein the coating, the core, or the absorption component, or any combination thereof, is designed in advance to absorb specific energy to rupture the particle, releasing the variable appearance material.

34. The particle of claim 32, wherein the coating, the core, or the absorption component, or any combination thereof, is designed in advance to absorb specific energy to alter variable appearance properties of the particle.

35. The particle of claim 32, wherein the specific energy is electromagnetic radiation.

36. The particle of claim 35, wherein the specific energy is infrared or near-infrared radiation.

37. The particle of claim 35, wherein the specific energy is ultraviolet or high-intensity visible radiation.

38. The particle of claim 29, wherein the coating comprises a metal oxide, silica, glass, fluorocarbon resin, organic polymer, wax, or any combination thereof.

39. The particle of claim 29, wherein the coating is substantially visibly transparent and absorbs near-infrared radiation.

40. The particle of claim 32, wherein the absorption component is selected from the group consisting of colored filter glass, graphite, carbon, a metal oxide, an acrylate polymer, a urethane polymer, silicon, germanium, a metal, an organo-metallic crystal, or a semiconductor material.

41. The particle of claim 32, wherein the coating comprises the absorption component that absorbs near-infrared radiation.

42. The particle of claim 39, wherein the coating comprises a material that absorbs the near-infrared radiation.

43. The particle of claim 29, wherein the coating comprises pores of a size sufficient to allow the variable appearance material to leach out of the particle.

44. The particle of claim 29, wherein multiple cores are enveloped within the coating of a single particle.

45. The particle of claim 29, wherein the coating comprises from about 10 to 95 percent of the volume of the particle.

46. The particle of claim 29, further comprising a sub-particle that comprises a neutralizing agent, which if released from the sub-particle upon exposure of the particle to specific energy, can neutralize variable appearance properties of the particle.

47. The particle of claim 46, wherein the specific energy is heat or electromagnetic radiation.

48. The particle of claim 46, wherein the variable appearance material is pH-sensitive and the neutralizing agent is an acid, a base, or a buffer that can effect a pH transition that neutralizes a variable appearance property of the particle.

49. The particle of claim 1, wherein the variable appearance material is photobleachable, and exposure of the particle to electromagnetic energy renders the particle substantially undetectable.

50. The particle of claim 1, wherein the variable appearance material is thermolabile, and exposure of the particle to thermal energy renders the particle substantially undetectable.

51. The particle of claim 1, wherein the variable appearance material has a refractive index sufficient for the particle to exhibit retro-reflection.

52. The particle of claim 51, wherein the particle has a spherical, corner cube, cubic crystal, or cubic crystal fragment shape.

53. The particle of claim 52, comprising a sphere with a diameter of greater than about a wavelength of light in a tissue to be marked, wherein said variable appearance material has a refractive index of at least about 1.6.

54. The particle of claim 53, wherein the refractive index is from about 1.6 to about 2.4.

55. The particle of claim 53, wherein the diameter of the sphere is from about one to about ten times the wavelength.

56. The particle of claim 53, wherein the tissue to be marked is dermis.

57. The particle of claim 53, wherein the tissue to be marked is selected from the group consisting of skin, iris, sclera, dentin, muscles, tendons, fingernails, toenails, tissue beneath fingernails, tissue beneath toenails, tissue inside the mouth, or tissue lining internal body passages.

58. The particle of claim 51, wherein the variable appearance material is indispersible.

59. A tissue marking ink comprising the particle of claim 1 and a liquid carrier.

60. The ink of claim 59, wherein the carrier comprises alcohol, water, or glycerin, or any combination thereof.

61. A method of applying to a tissue a variable appearance marking, the method comprising:
providing a particle comprising a variable appearance material, wherein the particle comprises a variable appearance material that varies in appearance via frequency up-conversion, retro-reflection, metachromasia or a change in an oxidation state; and
implanting the particle into the tissue.

62. The method of claim 61, comprising implanting the particle in skin, iris, sclera, dentin, fingernails, toenails, muscles, tendons, tissue beneath fingernails, tissue beneath toenails, tissue inside the mouth, or tissue lining internal body passages.

63. A method of changing and/or removing a variable appearance tissue marking with variable appearance properties that can be altered by application of a specific energy, wherein said marking is created by implanting into tissue a particle comprising a variable appearance material, the method comprising exposing said marking to the specific energy for a time sufficient to alter said variable appearance properties of the marking.

64. The method of claim 63, comprising exposing the marking to the specific energy to render the marking substantially undetectable.

65. The method of claim 63, comprising exposing the marking to the specific energy to rupture the particle and release the variable appearance material.

66. The method of claim 63, comprising exposing the marking to the specific energy to change the marking.

67. The method of claim 63, comprising applying the specific energy at a wavelength, at an intensity, or for a duration, or any combination thereof, insufficient to completely remove or change the variable appearance of the marking, thereby partially removing and/or changing the marking.

68. The method of claim 63, comprising exposing the marking to the specific energy to heat and alter the variable appearance material.

69. The method of claim 63, wherein the specific energy is electromagnetic radiation.

70. The method of claim 69, wherein the specific energy is near-ultraviolet or high-intensity visible radiation.

71. The method of claim 69, wherein the specific energy is infrared or near-infrared radiation.

72. The particle of claim 69, wherein the variable appearance material comprises a multi-photon photobleachable material.

73. The particle of claim 72, wherein the multi-photon photobleachable material comprises a two-photon photobleachable material.

74. The particle of claim 73, wherein the two-photon photobleachable material comprises a benzophenone, a ketone or a radical generator.

75. The particle of claim 73, wherein the specific energy is electromagnetic radiation below about 300 mm.
